SEQUENCE LISTING (87 sequences)

SEQ ID NO: 1-3 - skipped (3 entries)

SEQ ID NO: 4 - DNA, 10 bp, synthetic construct
  source 1..10: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttt

SEQ ID NO: 5 - DNA, 11 bp, synthetic construct
  source 1..11: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttt

SEQ ID NO: 6 - DNA, 14 bp, synthetic construct
  source 1..14: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 12: mod_base = OTHER; note = Deoxyuridine
  modified_base 13: mod_base = OTHER; note = Deoxyuridine
tttttttttttttt

SEQ ID NO: 7 - skipped

SEQ ID NO: 8 - DNA, 10 bp, synthetic construct
  source 1..10: mol_type = other DNA; note = DNA, artificial, single stranded
  modified_base 2: mod_base = OTHER; note = Deoxyuridine
  modified_base 3: mod_base = OTHER; note = Deoxyuridine
  modified_base 9: mod_base = OTHER; note = Deoxyuridine
  modified_base 10: mod_base = OTHER; note = Deoxyuridine
tttttttttt

SEQ ID NO: 9 - DNA, 10 bp, synthetic construct
  source 1..10: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
  modified_base 8: mod_base = OTHER; note = Deoxyuridine
  modified_base 9: mod_base = OTHER; note = Deoxyuridine
  modified_base 10: mod_base = OTHER; note = Deoxyuridine
tttttttttt

SEQ ID NO: 10 - DNA, 15 bp, synthetic construct
  source 1..15: mol_type = other DNA; note = DNA, artificial, single stranded
aacggaggaggagga

SEQ ID NO: 11 - DNA, 19 bp, synthetic construct
  source 1..19: mol_type = other DNA; note = DNA, artificial, single stranded
aacggaggaggaggacgta

SEQ ID NO: 12 - DNA, 16 bp, synthetic construct
  source 1..16: mol_type = other DNA; note = DNA, artificial, single stranded
taacggaggaggagga

SEQ ID NO: 13 - DNA, 15 bp, synthetic construct
  source 1..15: mol_type = other DNA; note = DNA, artificial, single stranded
aacggaggaggagga

SEQ ID NO: 14-17 - skipped (4 entries)

SEQ ID NO: 18 - DNA, 10 bp, synthetic construct
  source 1..10: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttt

SEQ ID NO: 19 - DNA, 11 bp, synthetic construct
  source 1..11: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttt

SEQ ID NO: 20 - DNA, 14 bp, synthetic construct
  source 1..14: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 12: mod_base = OTHER; note = Deoxyuridine
  modified_base 13: mod_base = OTHER; note = Deoxyuridine
tttttttttttttt

SEQ ID NO: 21 - skipped

SEQ ID NO: 22 - DNA, 10 bp, synthetic construct
  source 1..10: mol_type = other DNA; note = DNA, artificial, single stranded
  modified_base 2: mod_base = OTHER; note = Deoxyuridine
  modified_base 3: mod_base = OTHER; note = Deoxyuridine
  modified_base 9: mod_base = OTHER; note = Deoxyuridine
  modified_base 10: mod_base = OTHER; note = Deoxyuridine
tttttttttt

SEQ ID NO: 23 - DNA, 10 bp, synthetic construct
  source 1..10: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
  modified_base 8: mod_base = OTHER; note = Deoxyuridine
  modified_base 9: mod_base = OTHER; note = Deoxyuridine
  modified_base 10: mod_base = OTHER; note = Deoxyuridine
tttttttttt

SEQ ID NO: 24 - DNA, 25 bp, synthetic construct
  source 1..25: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttaacggaggaggagga

SEQ ID NO: 25 - DNA, 24 bp, synthetic construct
  source 1..24: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
tttttttttaacggaggaggagga

SEQ ID NO: 26 - DNA, 24 bp, synthetic construct
  source 1..24: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
tttttttttaacggaggaggagga

SEQ ID NO: 27 - DNA, 24 bp, synthetic construct
  source 1..24: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
tttttttttaacggaggaggagga

SEQ ID NO: 28 - DNA, 24 bp, synthetic construct
  source 1..24: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
tttttttttaacggaggaggagga

SEQ ID NO: 29 - DNA, 25 bp, synthetic construct
  source 1..25: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttaacggaggaggagga

SEQ ID NO: 30 - DNA, 25 bp, synthetic construct
  source 1..25: mol_type = other DNA; note = DNA, artificial, single stranded
  modified_base 2: mod_base = OTHER; note = Deoxyuridine
  modified_base 3: mod_base = OTHER; note = Deoxyuridine
  modified_base 8: mod_base = OTHER; note = Deoxyuridine
  modified_base 9: mod_base = OTHER; note = Deoxyuridine
ttttttttttaacggaggaggagga

SEQ ID NO: 31 - DNA, 25 bp, synthetic construct
  source 1..25: mol_type = other DNA; note = DNA, artificial, single stranded
  modified_base 2: mod_base = OTHER; note = Deoxyuridine
  modified_base 3: mod_base = OTHER; note = Deoxyuridine
  modified_base 9: mod_base = OTHER; note = Deoxyuridine
  modified_base 10: mod_base = OTHER; note = Deoxyuridine
tttttttttttaacggaggaggagg

SEQ ID NO: 32 - DNA, 25 bp, synthetic construct
  source 1..25: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
  modified_base 8: mod_base = OTHER; note = Deoxyuridine
  modified_base 9: mod_base = OTHER; note = Deoxyuridine
  modified_base 10: mod_base = OTHER; note = Deoxyuridine
tttttttttttaacggaggaggagg

SEQ ID NO: 33 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagga

SEQ ID NO: 34 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagga

SEQ ID NO: 35 - DNA, 24 bp, synthetic construct
  source 1..24: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
tttttttttaacggaggaggagga

SEQ ID NO: 36 - DNA, 24 bp, synthetic construct
  source 1..24: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
tttttttttaacggaggaggagga

SEQ ID NO: 37 - DNA, 25 bp, synthetic construct
  source 1..25: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttaacggaggaggagga

SEQ ID NO: 38 - DNA, 24 bp, synthetic construct
  source 1..24: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
tttttttttaacggaggaggagga

SEQ ID NO: 39 - DNA, 25 bp, synthetic construct
  source 1..25: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
ttttttttttaacggaggaggagga

SEQ ID NO: 40 - DNA, 25 bp, synthetic construct
  source 1..25: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
  modified_base 9: mod_base = OTHER; note = Deoxyuridine
ttttttttttaacggaggaggagga

SEQ ID NO: 41 - DNA, 25 bp, synthetic construct
  source 1..25: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
  modified_base 9: mod_base = OTHER; note = Deoxyuridine
ttttttttttaacggaggaggagga

SEQ ID NO: 42 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagga

SEQ ID NO: 43 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagga

SEQ ID NO: 44 - DNA, 15 bp, synthetic construct
  source 1..15: mol_type = other DNA; note = DNA, artificial, single stranded
aacggaggaggagga

SEQ ID NO: 45 - DNA, 15 bp, synthetic construct
  source 1..15: mol_type = other DNA; note = DNA, artificial, single stranded
aacggaggaggagga

SEQ ID NO: 46 - DNA, 15 bp, synthetic construct
  source 1..15: mol_type = other DNA; note = DNA, artificial, single stranded
aacggaggaggagga

SEQ ID NO: 47 - DNA, 15 bp, synthetic construct
  source 1..15: mol_type = other DNA; note = DNA, artificial, single stranded
aacggaggaggagga

SEQ ID NO: 48 - DNA, 16 bp, synthetic construct
  source 1..16: mol_type = other DNA; note = DNA, artificial, single stranded
taacggaggaggagga

SEQ ID NO: 49 - DNA, 16 bp, synthetic construct
  source 1..16: mol_type = other DNA; note = DNA, artificial, single stranded
taacggaggaggagga

SEQ ID NO: 50 - DNA, 16 bp, synthetic construct
  source 1..16: mol_type = other DNA; note = DNA, artificial, single stranded
taacggaggaggagga

SEQ ID NO: 51 - DNA, 24 bp, synthetic construct
  source 1..24: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttaacggaggaggagga

SEQ ID NO: 52 - DNA, 24 bp, synthetic construct
  source 1..24: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttaacggaggaggagga

SEQ ID NO: 53 - DNA, 15 bp, synthetic construct
  source 1..15: mol_type = other DNA; note = DNA, artificial, single stranded
aacggaggaggagga

SEQ ID NO: 54 - DNA, 19 bp, synthetic construct
  source 1..19: mol_type = other DNA; note = DNA, artificial, single stranded
aacggaggaggaggacgta

SEQ ID NO: 55 - DNA, 16 bp, synthetic construct
  source 1..16: mol_type = other DNA; note = DNA, artificial, single stranded
taacggaggaggagga

SEQ ID NO: 56 - DNA, 15 bp, synthetic construct
  source 1..15: mol_type = other DNA; note = DNA, artificial, single stranded
aacggaggaggagga

SEQ ID NO: 57 - protein, 305 aa, synthetic construct
  source 1..305: note = Polypeptide, artificial
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

SEQ ID NO: 58 - protein, 293 aa, synthetic construct
  source 1..293: note = Polypeptide, artificial
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 59 - protein, 10 aa, synthetic construct
  source 1..10: note = Polypeptide, artificial
AHIVMVDAYK

SEQ ID NO: 60 - protein, 10 aa, synthetic construct
  source 1..10: note = Polypeptide, artificial
GGSSGGSSGG

SEQ ID NO: 61 - protein, 13 aa, synthetic construct
  source 1..13: note = Polypeptide, artificial
AHIVMVDAYKPTK

SEQ ID NO: 62 - protein, 8 aa, synthetic construct
  source 1..8: note = Polypeptide, artificial
KGHHHHHH

SEQ ID NO: 63 - protein, 31 aa, synthetic construct
  source 1..31: note = Polypeptide, artificial
GGSSGGSSGGAHIVMVDAYKPTKKGHHHHHH

SEQ ID NO: 64 - DNA, 594 bp, synthetic construct
  source 1..594: mol_type = other DNA; note = DNA, artificial, double stranded
cagtcagtagagagagattggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaatctctctcaaaaacggaggaggaggacagtcagtagagagagattgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaatctctctcaaaaacggaggaggagga

SEQ ID NO: 65 - DNA, 594 bp, synthetic construct
  source 1..594: mol_type = other DNA; note = DNA, artificial, double stranded
cagtcagtagagagagattgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaatctctctcaaaaacggaggaggaggacagtcagtagagagagattggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaatctctctcaaaaacggaggaggagga

SEQ ID NO: 66 - DNA, 25 bp, synthetic construct
  source 1..25: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttaacggaggaggagga

SEQ ID NO: 67 - DNA, 25 bp, synthetic construct
  source 1..25: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttaacggaggaggagga

SEQ ID NO: 68 - DNA, 24 bp, synthetic construct
  source 1..24: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttaacggaggaggagga

SEQ ID NO: 69 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
  modified_base 10: mod_base = OTHER; note = Deoxyuridine
tttttttttttaacggaggaggagga

SEQ ID NO: 70 - DNA, 25 bp, synthetic construct
  source 1..25: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
  modified_base 9: mod_base = OTHER; note = Deoxyuridine
ttttttttttaacggaggaggagga

SEQ ID NO: 71 - DNA, 25 bp, synthetic construct
  source 1..25: mol_type = other DNA; note = DNA, artificial, single stranded
  modified_base 4: mod_base = OTHER; note = Deoxyuridine
  modified_base 5: mod_base = OTHER; note = Deoxyuridine
  modified_base 6: mod_base = OTHER; note = Deoxyuridine
  modified_base 9: mod_base = OTHER; note = Deoxyuridine
ttttttttttaacggaggaggagga

SEQ ID NO: 72 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagga

SEQ ID NO: 73 - DNA, 23 bp, synthetic construct
  source 1..23: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttaacggaggaggagga

SEQ ID NO: 74 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagga

SEQ ID NO: 75 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagga

SEQ ID NO: 76 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttaaacggaggaggagga

SEQ ID NO: 77 - DNA, 25 bp, synthetic construct
  source 1..25: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
ttttttttttaacggaggaggagga

SEQ ID NO: 78 - DNA, 25 bp, synthetic construct
  source 1..25: mol_type = other DNA; note = DNA, artificial, single stranded
  modified_base 2: mod_base = OTHER; note = Deoxyuridine
  modified_base 3: mod_base = OTHER; note = Deoxyuridine
  modified_base 8: mod_base = OTHER; note = Deoxyuridine
  modified_base 9: mod_base = OTHER; note = Deoxyuridine
ttttttttttaacggaggaggagga

SEQ ID NO: 79 - DNA, 26 bp, synthetic construct
  source 1..26: mol_type = other DNA; note = DNA, artificial, single stranded
  modified_base 2: mod_base = OTHER; note = Deoxyuridine
  modified_base 3: mod_base = OTHER; note = Deoxyuridine
  modified_base 9: mod_base = OTHER; note = Deoxyuridine
  modified_base 10: mod_base = OTHER; note = Deoxyuridine
tttttttttttaacggaggaggagga

SEQ ID NO: 80 - DNA, 16 bp, synthetic construct
  source 1..16: mol_type = other DNA; note = DNA, artificial, single stranded
taacggaggaggagga

SEQ ID NO: 81 - DNA, 16 bp, synthetic construct
  source 1..16: mol_type = other DNA; note = DNA, artificial, single stranded
taacggaggaggagga

SEQ ID NO: 82 - DNA, 30 bp, synthetic construct
  source 1..30: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 12: mod_base = OTHER; note = Deoxyuridine
  modified_base 13: mod_base = OTHER; note = Deoxyuridine
tttttttttttttttaacggaggaggagga

SEQ ID NO: 83 - DNA, 26 bp, synthetic construct
  source 1..26: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagga

SEQ ID NO: 84 - DNA, 25 bp, synthetic construct
  source 1..25: mol_type = other DNA; note = DNA, artificial, single stranded
  modified_base 5: mod_base = OTHER; note = Deoxyuridine
  modified_base 6: mod_base = OTHER; note = Deoxyuridine
  modified_base 7: mod_base = OTHER; note = Deoxyuridine
tttttttttttaacggaggaggagg

SEQ ID NO: 85 - DNA, 15 bp, synthetic construct
  source 1..15: mol_type = other DNA; note = DNA, artificial, single stranded
aacggaggaggagga

SEQ ID NO: 86 - DNA, 15 bp, synthetic construct
  source 1..15: mol_type = other DNA; note = DNA, artificial, single stranded
aacggaggaggagga

SEQ ID NO: 87 - DNA, 15 bp, synthetic construct
  source 1..15: mol_type = other DNA; note = DNA, artificial, single stranded
aacggaggaggagga

(19) United States
(12) Patent Application Publication
(10) Pub. No.: US 2023/0159999 A1
(43) Pub. Date: May 25, 2023

(54) COMPOSITIONS THAT REDUCE TEMPLATE THREADING INTO A NANOPORE

(71) Applicant: Roche Sequencing Solutions, Inc., Pleasanton, CA (US)

(72) Inventors: Aruna Ayer, Santa Clara, CA (US); David Beyer, Santa Clara, CA (US); Peter Crisalli, Mountain View, CA (US); Kirti Dhiman, Sunnyvale, CA (US); Helen Franklin, San Jose, CA (US); Swathi Chakravarthy, Santa Clara, CA (US); Omid Khakshoor, Lanthrop, CA (US); Blake Langdon, Santa Clara, CA (US); Meng Taing, Hayward, CA (US); Adolfo Vargas, San Mateo, CA (US)

(21) Appl. No.: 17/817,480

(22) Filed: Aug. 4, 2022

Related U.S. Application Data
(63) Continuation of application No. PCT/EP2021/052669, filed on Feb. 4, 2021.
(60) Provisional application No. 62/971,078, filed on Feb. 6, 2020.

(51) Int. Cl. C12Q 1/6869

(52) U.S. Cl. CPC C12Q 1/6869

(57) ABSTRACT

This application discloses compositions comprising primer compounds that reduce or block deleterious threading into a nanopore of nucleic acid strands displaced by a nanopore-linked polymerase, for example during the use of a nanopore device for nucleic acid sequencing. Also disclosed are methods for using the compositions to reduce deleterious threading events during nanopore-based nucleic acid detection techniques, such as nanopore sequencing.

### FIELD

This application relates to compositions that reduce or block deleterious template threading during strand polymerization by nanopore-linked polymerase, and methods for using the composition in nanopore-based nucleic acid detection techniques, such as nanopore sequencing.

### BACKGROUND

Nanopore single-molecule sequencing-by-synthesis (“SBS”) uses a polymerase (or other strand-extending enzyme) covalently linked to nanopore to synthesize a DNA strand complementary to a target sequence template (i.e., a copy strand) and concurrently detect the identity of each nucleotide monomer as it is added it to that growing strand. See e.g., US Pat. Publ. Nos. 2013/0244340 A1, 2013/0264207 A1, 2014/0134616 A1, 2015/0368710 A1, and 2018/0057870 A1, and published International Application WO 2019/166457 A1. Each added nucleotide monomer is detected by monitoring signals due to changes in ion flow through the nanopore that is located adjacent to the polymerase active site as the copy strand is synthesized. Obtaining an accurate, reproducible ion flow signal requires positioning the polymerase active site near the nanopore so as to allow a tag moiety attached to each added nucleotide to enter and alter the ion flow through the nanopore. For optimal performance, the tag moiety should reside in the nanopore for a sufficient amount of time to provide for a detectable, identifiable, and reproducible signal associated with altering ion flow through the nanopore (relative to the baseline “open current” flow), such that the specific nucleotide associated with the tag can be distinguished unambiguously from the other tagged nucleotides in the SBS solution.

Kumar et al., (2012) “PEG-Labeled Nucleotides and Nanopore Detection for Single Molecule DNA Sequencing by Synthesis,” Scientific Reports, 2:684; DOI: 10.1038/srep00684, describes using a nanopore to distinguish four different length PEG-coumarin tags attached via a terminal 5′-phosphoramidate to a dG nucleotide, and separately demonstrates efficient and accurate incorporation of these four PEG-coumarin tagged dG nucleotides by DNA polymerase. See also, US Patent Application Publications 2013/0244340 A1, 2013/0264207 A1, 2014/0134616 A1, 2015/0368710 A1, and 2018/0057870 A1.

WO 2013/154999 and WO 2013/191793 describe the use of tagged nucleotides for nanopore SBS and disclose the possible use of a single nucleotide attached to a single tag comprising branched PEG chains.

WO 2015/148402 describes the use of tagged nucleotides for nanopore SBS comprising a single nucleotide attached to a single tag, wherein the tag comprises any of a range of oligonucleotides (or oligonucleotide analogues) that have lengths of 30 monomer units or longer.

U.S. Pat. No. 9,410,172 B2 describes methods and kits for isothermal nucleic acid amplifications that use an oligocation-oligonucleotide conjugate primer to amplify a target nucleic acid. The disclosed methods employ a strand displacing DNA polymerase and a polyamine oligonucleotide conjugate primer.

“Wide-pore” mutants of the nanopore alpha-hemolysin (“α-HL”) have been developed which exhibit a longer lifetime when used in nanopore devices and exposed to the electrochemical conditions used in conducting high-throughput nanopore sequencing. See e.g., WO 2019/166457 A1, published Sep. 6, 2019. The longer nanopore lifetime provides greater read-lengths and overall accuracy in sequencing. Structurally, the wide-pore mutants are engineered to effectively eliminate the naturally occurring constriction site (i.e., narrowest portion of pore) that is located at a depth of approximately 40 angstroms from the cis opening of the pore, and which has a diameter of approximately 10 angstroms in diameter. The wide-pore mutations create a new constriction site located deeper into the pore, approximately 65 angstroms from the cis opening, and which is wider—approximately 13 angstroms in diameter.

Despite advantages for improved lifetimes, wide-pore α-HL nanopores still suffer from deleterious stoppage events when used in nanopore SBS. These deleterious events are understood to result from the template strand threading into the nearby nanopore and interfering with further detection of tag moieties as polymerization proceeds. This template threading phenomenon results in abbreviated sequence reads and overall lower throughput for nanopore SBS.

Accordingly, there remains a need for compositions and methods that reduce or prevent deleterious template threading when using nanopores and thereby result in improved efficiency of high-throughput nanopore detection techniques, such as nucleic acid SBS.

### SUMMARY

In at least one embodiment, the present disclosure provides a composition comprising a compound of formula (I):

5′-[Blocking Moiety]-[Primer]-3′ (I)

wherein, Blocking Moiety comprises a poly-cationic group, a bulky group, or a base-modified nucleoside, wherein the base-modified nucleoside comprises a poly-cationic group or a bulky group attached to the nucleoside base; and Primer comprises an oligonucleotide capable of priming polymerization of a copy strand by a polymerase linked to a nanopore.

In at least one embodiment of the composition, the compound of formula (I) comprises a compound selected from:
(a) a compound of formula (Ia):

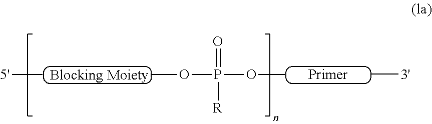

wherein, n is 1 to 10; and R is independently selected from O⁻, S⁻, CH₃, and H;
(b) a compound of formula (Ib):

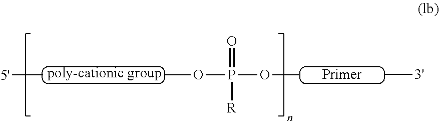

wherein, n is 1 to 10; and R is independently selected from O⁻, CH₃, and H;
c) A compound of formula (Ic):

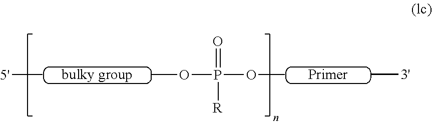

wherein, n is 1 to 10; and R is independently selected from O⁻, S⁻, CH₃, and H;
(d) a compound of formula (Id):

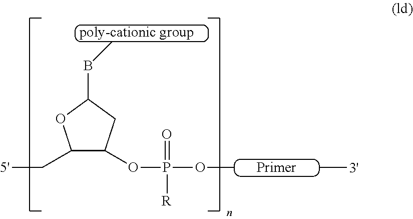

wherein, n is 1 to 10; B is a modified nucleobase; and R is independently selected from O⁻, S⁻, CH₃, and H; or
(e) The composition of claim 1, wherein the compound of formula (I) comprises a compound of formula (Ie):

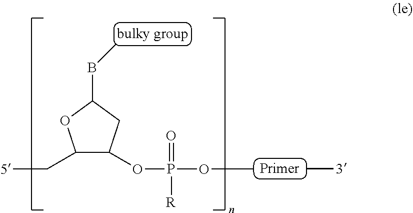

wherein, n is 1 to 10; B is a modified nucleobase; and R is independently selected from O⁻, S⁻, CH₃, and H.

In at least one embodiment of the composition, the compound of formula (I) further comprises a biotin tag attached to the 5′-end of the Blocking Moiety.

In at least one embodiment, the present disclosure provides a composition comprising a compound of formula (II):

5′-[Biotin Tag]-[Blocking Moiety]-[Primer]-3′ (II)

wherein, Biotin Tag comprises a biotin tag; Blocking Moiety comprises a poly-cationic group, a bulky group, or a base-modified nucleoside, wherein the base-modified nucleoside comprises a poly-cationic group or a bulky group attached to the nucleoside base; and Primer comprises an oligonucleotide capable of priming polymerization of a copy strand by a polymerase linked to a nanopore.

In at least one embodiment of the composition, the compound of formula (II) comprises a compound selected from:
(a) a compound of formula (IIa):

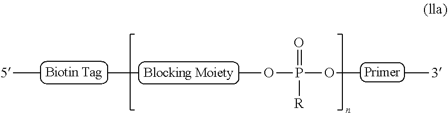

wherein, n is 1 to 10; and R is independently selected from O⁻, S⁻, CH₃, and H;
(b) a compound of formula (IIb):

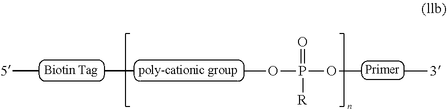

wherein, n is 1 to 10; and R is independently selected from O⁻, S⁻, CH₃, and H;
(c) a compound of formula (IIc):

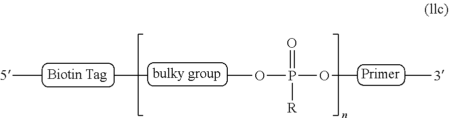

wherein, n is 1 to 10; and R is independently selected from O⁻, S⁻, CH₃, and H;
(d) a compound of formula (IId):

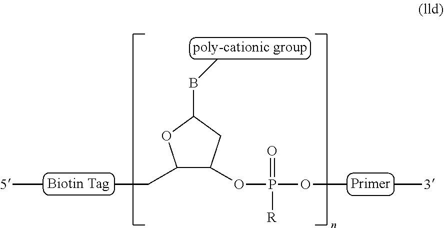

wherein, n is 1 to 10; B is a modified nucleobase; and R is independently selected from O⁻, S⁻, CH₃, and H; or
(e) a compound of formula (IIe):

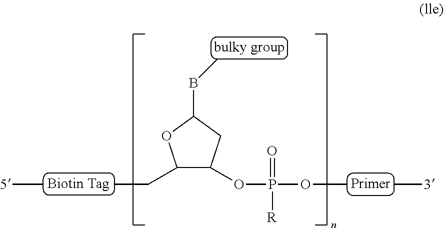

wherein, n is 1 to 10; B is a modified nucleobase; and R is independently selected from O⁻, S⁻, CH₃, and H.

In at least one embodiment of the composition comprising a compound of formula (II), the biotin tag comprises a structure of formula (III):

B-L-[(N)ₓ—(U)_{y}—(N)_{z}]_{w} (III)

wherein, B is biotin or desthiobiotin; L is a linker; N is a nucleotide; U is uracil; and x and z are at least 1; y is at least 3; and w is 0 or 1.

In at least one embodiment of the composition comprising a compound of formula (II), the biotin tag comprises a biotin moiety and a linker moiety or a desthiobiotin moiety and a linker moiety, wherein the linker moiety attaches to the 5′-end of the Blocking Moiety; optionally, wherein the linker moiety comprises an oligonucleotide; optionally, wherein the oligonucleotide comprises a sequence selected from: TTTTUUU (SEQ ID NO: 1); TTTTUUUT (SEQ ID NO: 2); TTTTUUUTT (SEQ ID NO: 3); TTTTUUUTTT (SEQ ID NO: 4); TTTTUUUTTTT (SEQ ID NO: 5); TTTTUUTITTUUT (SEQ ID NO: 6); TUUTTTTUU (SEQ ID NO:7); TUUTTTTTUU (SEQ ID NO: 8); and TTTTUUUUUU (SEQ ID NO: 9).

In at least one embodiment of the composition comprising a compound of formula (I) or formula (II), the Blocking Moiety comprises a poly-cationic group, wherein
(a) the poly-cationic group is selected from spermine, spermidine, [Phe(4-NO₂)-εLys-(Lys)₈], [Phe(4-NO₂)-εLys-(Lys)₁₂]. [(Lys)₈-εLys-Phe(4-NO₂)], [(Lys)₁₂-εLys-Phe(4-NO₂)], [PAMAM Gen1 amino], poly(ethylenediamine), poly(propylenediamine), poly(allylamine), an oligomer of a cationic amino acid, and an oligomer of a cationic aminoalkyl;
(b) the poly-cationic group is an oligomer of a cationic amino acid selected from an oligomer of lysine, ε-lysine, omithine, (aminoethyl)glycine, arginine, histidine, methyllysine, dimethyllysine, trimethyllysine, and/or aminoproline; and/or
(c) wherein the poly-cationic group is an oligomer of spermine groups; optionally, wherein the oligomer of spermine groups comprises an oligomer selected from: (spermine)₂, (spermine)₃, (spermine)₄, and (spermine)₅; optionally, wherein the spermine groups of the oligomer are phosphodiester-linked.

In at least one embodiment of the composition comprising a compound of formula (I) or formula (II), the Blocking Moiety comprises a bulky group, wherein
(a) the bulky group is selected from an aryl, arylalkyl, heteroaryl, heteroarylalkyl, cycloalkyl, heterocycloalkyl, and combination thereof;
(b) the bulky group is selected from pyrene, cholesteryl, beta-cyclodextrin, high poly(ethylene glycol) polymers, perylene, perylenediimides, and cucurbituril; and/or
(c) the bulky group is a phosphodiester-linked bulky group.

In at least one embodiment of the composition comprising a compound of formula (I) or formula (II). the Blocking Moiety comprises a base-modified nucleoside, wherein:
(a) the base-modification comprises a poly-cationic group selected from poly-lysine, poly-arginine, poly-histidine, poly-omithine, poly-(aminoethyl)glycine, poly-methyllysine, poly-dimethyllysine, poly-trimethyllysine, poly-aminoproline, and poly-ε-lysine; or
(b) the base-modification comprises a bulky group selected from perylene, cholesteryl, and beta-cyclodextrin.

In at least one embodiment of the composition the compound of formula (I) is selected from: 5′-(spermine)₂-[Primer]-3′; 5′-(spermine)₃-[Primer]-3′; 5′-(spermine)₄-[Primer]-3′; 5′-(spermine)₅-[Primer]-3′; 5′-(pyrene)₂-[Primer]-3′; 5′-(cholestyryl)-[Primer]-3′; 5′-[Phe(4-NO₂)-εLys-(Lys)₁₂]-[Primer]-3′; 5′-[(Lys)_{Q}-εLys-Phe(4-NO₂)]-[Primer]-3′; 5′-[(Lys)₁₂-εLys-Phe(4-NO₂)]-[Primer]-3′; 5′-[PAMAM Gen1 amino]-[Primer]-3′; and 5′-(perylene-dU)-[Primer]-3′.

In at least one embodiment of the composition comprising a compound of formula (I) or formula (II), the Primer comprises:
(a) an oligonucleotide of at least 9-mer, at least 12-mer, or at least 15-mer;
(b) a locked nucleic acid;
(c) a linkage selected from a phosphorothioate, a methyl phosphonate, a phosphotriester, a phosphoramide, and a boronophosphate; and/or
(d) a sequence selected from: AACGGAGGAGGAGGA (SEQ ID NO: 10),
   AACGGAGGAGGAGGACGTA (SEQ ID NO:11),
   TAA\hat{ }CGGA\hat{ }GGA\hat{ }GGA\hat{ }GGA-3′ (SEQ ID NO: 12), and
   AACGGAGGAGGA*G*G*A-3′ (SEQ ID NO: 13).

In at least one embodiment of the composition comprising a compound of formula (I) or formula (II), the compound is selected from:

5′-(Biotin)-(Sp18)-TTTTUUUTT-(spermine)-
TAACGGAGGAGGAGGA-3′;
5′-(Biotin)-(Sp18)-TTTUUUTT-(spermine)₂-
TAACGGAGGAGGAGGA-3′;
5′-(Biotin)-(Sp18)-TTTUUUTT-(spermine)₃-
TAACGGAGGAGGAGGA-3′;
5′-(Biotin)-(Sp18)-TTTUUUTT-(spermine)₄-
TAACGGAGGAGGAGGA-3′;
5′-(Biotin)-(Sp18)-TTTUUUTT-(spermine)₅-
TAACGGAGGAGGAGGA-3′;
5′-(Biotin)-(Sp18)-TTTTUUUTT-(spermine)₅-
TAACGGAGGAGGAGGA-3′;
5′-(Biotin)-(Sp18)-TUUTTTTUU-(spermine)₃-
TAACGGAGGAGGAGGA-3′;
5′-(Biotin)-(Sp18)-TUUTTTTTUU-(spermine)₂-
TAACGGAGGAGGAGGA-3′;
5′-(Biotin)-(Sp18)-TTTTUUUUUU-(spermine)₂-
TAACGGAGGAGGAGGA-3′;
5′-(Biotin)-(Sp18)-TTTTUUUTTT-(ethylendiamine)5-
TAACGGAGGAGGAGGA-3′;
5′-(Biotin)-(Sp18)-TTTTUUUTTT-(ethylendiamine)6-
TAACGGAGGAGGAGGA-3′;
5′-(Biotin)-(Sp18)-TTTUUUTT-(pyrene)₂-
TAACGGAGGAGGAGGA-3′;
5′-(Biotin)-(Sp18)-TTTUUUTT-(cholesteryl)-
TAACGGAGGAGGAGGA-3′;
5′-Biotin-(Sp18)-TTTTUUUTTT-(T*[Phe(4-NO₂)-
(ϵ-Lys)-(Lys)₈])-AACGGAGGAGGAGGA-3′
5′-(Biotin)-(Sp18)-TTTUUUTT-(T*[Phe(4-NO₂)-
(ϵ-Lys)-(Lys)₁₂])-TAACGGAGGAGGAGGA-3′;
5′-(Biotin)-(Sp18)-TTTUUUTT-(T*-[PAMAM Gen1
amino))-TAACGGAGGAGGAGGA-3′;
5′-(Biotin)-(Sp18)-TTTUUUTT-(dU-perylene)-
TAACGGAGGAGGAGGA-3′;
5′-(Biotin)2-(Sp18)-TTTUUUTT-(dU-perylene)-
TAACGGAGGAGGAGGA-3′
5′-(Biotin)-(Sp18)-TTTTUUUTTT-([Phe(4-NO₂)-
(ϵ-Lys)-(Lys)₈]-T\hat{ })-AACGGAGGAGGA*G*G*A-3′;
5′-(Biotin)-(Sp18)-TTTTUUUTTT-[dbco-Phe(4-NO₂)-
(ϵ-Lys)-(Lys)₈)-T\hat{ })-AACGGAGGAGGA*G*G*A-3′;
5′-(DesthiobiotinTEG)-(Sp18)2-(T*-[Phe(4-NO₂)-
(ϵ-Lys)-(Lys)₈])-AACGGAGGAGGAGGA-3′;
5′-(DesthiobiotinTEG)-(Sp18)3-(T*-[Phe(4-NO₂)-
(ϵ-Lys)-(Lys)₈])-AACGGAGGAGGAGGA-3′;
5′-(DesthiobiotinTEG)-(Sp18)3-(T*-[Phe(4-NO₂)-
(ϵ-Lys)-(Lys)₁₂])-AACGGAGGAGGAGGA-3′;
5′-(DesthiobiotinTEG)-(Sp18)₃-(spermine)₂-
TAACGGAGGAGGAGGA-3′;
5′-(DesthiobiotinTEG)-(Sp18)₃-(spermine)₃-
TAACGGAGGAGGAGGA-3′;
5′-(DesthiobiotinTEG)-(Sp18)₃-(spermine)₄-
TAACGGAGGAGGAGGA-3′;
5′-(DesthiobiotinTEG)-(Sp18)₃-(spermine)₅-
TAACGGAGGAGGAGGA-3′;
5′-(Biotin)-(Sp18)-TTTTUUUT-(spermine)₂-
TAA\hat{ }CGGA\hat{ }GGA\hat{ }GGA\hat{ }GGA-3′
or
5′-(Biotin)-(Sp18)-TTTTUUUT-(spermine)₂-
TAA\hat{ }CGGA\hat{ }GGA\hat{ }GGA\hat{ }GGA-3′

In at least one embodiment of the composition comprising a compound of formula (I) or formula (II), the composition further comprises a polymerase linked to a nanopore; optionally, wherein the polymerase is a Pol6 polymerase; optionally, wherein the nanopore is a wide-pore mutant α-HL nanopore; optionally, wherein the wide-pore mutant α-HL nanopore is selected from P-01, P-02, P-03, P-04, P-05, P-05, P-06, P-07, P-08, P-09, P-10, P-11, and P-12.

In at least one embodiment of the composition comprising a compound of formula (I) or formula (II), the compound is:
(a) capable of priming polymerization of a copy strand by a polymerase linked to a nanopore with a read length of at least 1000 bp, at least 1500 bp, at least 2000 bp, at least 2500 bp, or more; and/or
(b) capable of priming polymerization of a copy strand by a polymerase linked to a nanopore with a template threading rate of less than 50%, less than 40%, less than 30%, less than 20%, less than 10%, or less.

In at least one embodiment, the present disclosure provides a nanopore composition comprising: a membrane having an electrode on a cis side and a trans side of the membrane; a nanopore with its pore extending through the membrane; an active polymerase situated adjacent to the nanopore; an electrolyte solution comprising ions in contact with both electrodes; and a compound of formula (I) and/or formula (II); optionally, wherein the nanopore is a wide-pore mutant α-HL nanopore, and/or polymerase is Pol6 polymerase.

In at least one embodiment, the present disclosure provides a kit comprising: a nanopore device comprising a membrane having an electrode on a cis side and a trans side of the membrane, a nanopore with its pore extending through the membrane, and an active polymerase situated adjacent to the nanopore; a set of four tagged nucleotides; and a composition comprising a compound of formula (I) or formula (II).

In at least one embodiment, the present disclosure provides a method for determining the sequence of a nucleic acid comprising: (a) providing a nanopore composition comprising: a membrane, an electrode on the cis side and the trans side of the membrane, a nanopore with its pore extending through the membrane, an active polymerase situated adjacent to the nanopore, an electrolyte solution comprising ions in contact with both electrodes, and a composition of comprising a compound of formula (I) or formula (II); (b) contacting the nanopore composition of (a) with: (i) a nucleic acid; and (ii) a set of four tagged nucleotides, each capable of acting as polymerase substrate and each linked to a different tag which results in a different altering of the flow of ions through the nanopore when the tag enters the nanopore: and (c) detecting the different altering of the flows of ions resulting from the entry of the different tags in the nanopore over time and correlating to each of the different compounds incorporated by the polymerase which are complementary to the nucleic acid sequence, and thereby determining the nucleic acid sequence.

### BRIEF DESCRIPTION OF THE DRAWINGS

FIG. 1 depicts an exemplary CuAAC reaction scheme for modifying an ethynyl-dU nucleoside unit within an oligonucleotide with a azido-perylene bulky group.

FIG. 2 depicts an exemplary CuAAC reaction for preparing the threading-blocker primer, 5′-(Biotin)-(Sp18)-TTTTUUUTTT-(T*-[Phe(4-NO₂)-εLys-(Lys)₈])-AACGGAGGAGGAGGA-3′, wherein the Blocking Moiety comprises a single dU nucleoside modified with an 8 carbon linker alkyne group that is further base-modified via CuAAC chemistry with the 8 lysine poly-cationic group, [Phe(4-NO₂)-εLys-(Lys)₈].

FIG. 3 depicts a schematic structure of a threading-blocker primer, 5′-(Biotin)-(Sp18)-TTTTUUUTTT-(T*-[Phe(4-NO₂)-εLys-(Lys)₁₂])-AACGGAGGAGGAGGA-3′, wherein the Blocking Moiety comprises a single T nucleoside that is base-modified via CuAAC chemistry with a 12 lysine poly-cationic group, [Phe(4-NO₂)-εLys-(Lys)₁₂], and a Biotin Tag attached to the 5′-end of the Blocking Moiety, wherein the Biotin Tag comprises a linker that includes an Sp18 spacer and a TTTTUUUTTT cleavable oligonucleotide.

FIG. 4 depicts a schematic structure of the threading-blocker primer, 5′-(Biotin)-(Sp18)-TTTTUUUTTT-(T*-[PAMAM Gen1 amino])-AACGGAGGAGGAGGA-3′, wherein the Blocking Moiety comprises a single T nucleoside that is base-modified via CuAAC chemistry with the poly-cationic “PAMAM Gen1 amino” group, and a Biotin Tag attached to the 5′-end of the Blocking Moiety, wherein the Biotin Tag comprises a linker that includes an Sp18 spacer and a TTTTUUUTTT cleavable oligonucleotide.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50” includes “2 to 25”, “5 to 20”, “25 to 50”, “1 to 10”, etc.

It is to be understood that both the foregoing general description, including the drawings, and the following detailed description are exemplary and explanatory only and are not restrictive of this disclosure.

### Definitions

“Nucleoside,” as used herein, refers to a molecular moiety that comprises a naturally occurring or a non-naturally occurring nucleobase attached to a sugar moiety (e.g., ribose or deoxyribose).

“Nucleotide,” as used herein refers to a nucleoside-5′-oligophosphate compound or a structural analog of a nucleoside-5′-oligophosphate. Exemplary nucleotides include, but are not limited to, nucleoside-5′-triphosphates (e.g., dATP, dCTP, dGTP, dTTP, and dUTP); nucleosides (e.g., dA, dC, dG, dT, and dU) with 5′-oligophosphate chains of 4 or more phosphates in length (e.g., 5′-tetraphosphosphate, 5′-pentaphosphosphate, 5′-hexaphosphosphate, 5′-heptaphosphosphate, 5′-octaphosphosphate); and structural analogs of nucleoside-5′-triphosphates that can have a modified nucleobase moiety (e.g., a substituted pyrimidine nucleobase such as 5-ethynyl-dU), a modified sugar moiety (e.g., an O-alkylated sugar, or a 2′-4′ “locked” ribose), and/or a modified oligophosphate moiety (e.g., an oligophosphate comprising a thio-phosphate, a methylene, and/or other bridges between phosphates).

“Nucleic acid,” as used herein, refers to a molecule of one or more nucleic acid subunits which comprise one of the nucleobases, adenine (A), cytosine (C), guanine (G), thymine (T), and uracil (U), or variants thereof. Nucleic acid can refer to a polymer of nucleotides (e.g., dAMP, dCMP, dGMP, dTMP), also referred to as a polynucleotide, and includes DNA, RNA, in both single and double-stranded form, and hybrids thereof.

“Oligonucleotide,” as used herein, refers to a molecular moiety that comprises an oligomer of nucleotides. It is intended that “oligonucleotide” can refer to molecular moiety comprising an oligomer of nucleotides that also includes one or more monomer units that are not nucleotides (e.g., spacers such as, SpC2, SpC3, dSp, Sp18, or large groups such as, spermine, pyrene). It is also intended that “oligonucleotide” can refer to a molecular moiety comprising phosphodiester linkages and/or other non-natural linkages (e.g., phosphorothioate, methyl phosphonate, phosphotriester, phosphoramide, boronophosphate) between monomer units.

“Oligophosphate,” as used herein, refers to a molecular moiety that comprises an oligomer of phosphate groups. For example, an oligophosphate can comprise an oligomer of from 2 to 20 phosphates, an oligomer of from 3 to 12 phosphates, an oligomer of from 3 to 9 phosphates.

“Polymerase,” as used herein, refers to any natural or non-naturally occurring enzyme or other catalyst that is capable of catalyzing a polymerization reaction, such as the polymerization of nucleotide monomers to form a nucleic acid polymer. The term polymerase encompasses a variety of strand-extending enzymes including, but not limited to, DNA polymerases, RNA polymerases, and reverse transcriptases. Exemplary polymerases that may be used in the compositions and methods of the present disclosure include the nucleic acid polymerases such as DNA polymerase (e.g., enzyme of class EC 2.7.7.7), RNA polymerase (e.g., enzyme of class EC 2.7.7.6 or EC 2.7.7.48), reverse transcriptase (e.g., enzyme of class EC 2.7.7.49), and DNA ligase (e.g., enzyme of class EC 6.5.1.1).

“Read length” as used herein refers to the number of nucleotides that a strand-extending enzyme, such as a polymerase, incorporates into a nucleic acid strand in a template-dependent manner prior to dissociation from the template.

“Template DNA molecule” and “template strand” are used interchangeably herein to refer to a strand of a nucleic acid molecule that is used by a strand-extending enzyme (e.g., DNA polymerase) to synthesize a complementary nucleic acid strand (or copy strand), for example, in a primer extension reaction.

“Template-dependent manner” as used herein refers to the extension of a primer molecule by a strand-extending enzyme (e.g., DNA polymerase) wherein the sequence of the newly synthesized strand is dictated by the well-known rules of complementary base pairing to the template strand (see, for example, Watson, J. D. et al., In: Molecular Biology of the Gene, 4th Ed., W. A. Benjamin, Inc., Menlo Park, Calif. (1987)).

“Primer” as used herein refers to an oligonucleotide, whether occurring naturally or produced synthetically, which is capable of acting as a point of initiation of template-dependent nucleic acid synthesis by a strand-extending enzyme (e.g., DNA polymerase) under conditions suitable for synthesis of a primer extension product complementary to the template strand (or copy strand), e.g., in the presence of nucleotides, in an appropriate buffer, and at a suitable temperature. Primer length can depend on the complexity of the target sequence of the template strand, primer oligonucleotides typically contain 15-25 nucleotides, although it may contain more or few nucleotides.

“Enzyme-nanopore complex” as used herein refers to a nanopore that is associated with, coupled with, or linked to a strand-extending enzyme, such as a DNA polymerase (e.g., variant Pol6 polymerase). In some embodiments, the nanopore can be reversibly or irreversibly bound to the strand-extending enzyme.

“Moiety,” as used herein, refers to part of a molecule.

“Linker,” as used herein, refers to any molecular moiety that provides a bonding attachment with some space between two or more molecules, molecular groups, and/or molecular moieties.

“Tag,” as used herein, refers to a moiety or part of a molecule that enables or enhances the ability to detect and/or identify, either directly or indirectly, a molecule or molecular complex, which is coupled to the tag. For example, the tag can provide a detectable property or characteristic, such as steric bulk or volume, electrostatic charge, electrochemical potential, optical and/or spectroscopic signature.

“Nanopore,” as used herein, refers to a pore, channel, or passage formed or otherwise provided in a membrane or other barrier material that has a characteristic width or diameter of about 1 angstrom to about 10,000 angstroms. A nanopore can be made of a naturally-occurring pore-forming protein, such as α-hemolysin from S. aureus, or a mutant or variant of a wild-type pore-forming protein, either non-naturally occurring (i.e., engineered) such as α-HL-C46, or naturally occurring. A membrane may be an organic membrane, such as a lipid bilayer, or a synthetic membrane made of a non-naturally occurring polymeric material. The nanopore may be disposed adjacent or in proximity to a sensor, a sensing circuit, or an electrode coupled to a sensing circuit, such as, for example, a complementary metal-oxide semiconductor (CMOS) or field effect transistor (FET) circuit.

“Wide-pore mutant,” as used herein, refers to a nanopore engineered to have a constriction site of about 13 angstroms diameter located at a depth of about 65 angstroms as measured from the widest portion of the cis side of the pore when it is embedded in a membrane. Exemplary wide-pore mutants include α-HL heptamers comprising a 6:1 ratio of mutant α-HL subunits as disclosed elsewhere herein.

“Nanopore-detectable tag” as used herein refers to a tag that can enter into, become positioned in, be captured by, translocate through, and/or traverse a nanopore and thereby result in a detectable change in current through the nanopore. Exemplary nanopore-detectable tags include, but are not limited to, natural or synthetic polymers, such as polyethylene glycol, oligonucleotides, polypeptides, carbohydrates, peptide nucleic acid polymers, locked nucleic acid polymers, any of which may be optionally modified with or linked to chemical groups, such as dye moieties, or fluorophores, that can result in detectable nanopore current changes.

“Ion flow,” as used herein, refers to the movement of ions, typically in a solution, due to an electromotive force, such as the potential between an anode and a cathode. Ion flow typically can be measured as current or the decay of an electrostatic potential.

“Ion flow altering,” as used herein in the context of nanopore detection, refers to the characteristic of resulting in a decrease or an increase in ion flow through a nanopore relative to the ion flow through the nanopore in its “open channel” (O.C.) state.

“Open channel current,” “O.C. current,” or “Background current” as used herein refers to the current level measured across a nanopore when a potential is applied and the nanopore is open (e.g., no tag is present in the nanopore).

“Tag current” as used herein refers to the current level measured across a nanopore when a potential is applied and a tag is present the nanopore. For example, depending on a tag's specific characteristics (e.g., overall charge, structure, etc.), the presence of the tag in a nanopore can decrease ion flow through the nanopore and thereby result in a decrease in measured tag current level.

### Detailed Description of Various Embodiments

A. Threading-Blocker Primer Compounds

The present disclosure provides compounds that have been optimized to reduce deleterious template threading when used as primers with strand extending enzymes (e.g., polymerases) located adjacent to nanopores (e.g., α-hemolysin). These compounds are useful with nanopore-based methods for the detection and/or sequencing of nucleic acids that utilize tagged nucleosides and a strand-extending enzyme, such as a polymerase, located adjacent to a nanopore.

Generally, nanopore-based nucleic acid detection and/or sequencing uses strand extending enzyme (e.g., Pol6 DNA polymerase) located adjacent to a membrane-embedded nanopore (e.g., α-HL) and a mixture of four nucleotide analogs (e.g., dA6P, dC6P, dG6P, and dT6P) that can be incorporated by the strand extending enzyme into a growing strand. Each nucleotide analog has a covalently attached tag moiety that provides an identifiable, and distinguishable signature when detected with a nanopore. The strand extending enzyme forms a complex a template nucleic acid strand and a primer and specifically binds to a tagged nucleotide analog that is complimentary to the template nucleic acid strand. The strand extending enzyme then catalytically couples (i.e., incorporates) the nucleotide moiety of the tagged nucleotide analog to the 3′-end of the primer. Completion of the catalytic incorporation event results in the release of the tag moiety which then passes through the adjacent nanopore. However, even before it undergoes catalytic process that releases it from the incorporated nucleotide, the tag moiety of a enters the pore of the membrane embedded nanopore. When the nanopore is under an applied potential, this entry of the tag moiety alters the ion flow through the nanopore and provides a detectable tag current signal.

A variety of nanopore systems comprising a strand extending enzyme adjacent to a membrane-embedded nanopore and methods for using them with primers and tagged nucleotides for nucleic acid sequencing are known in the art. See, for example, US Patent Application Publications 2009/0298072 A1, 2013/0244340 A1, 2013/0264207 A1, 2014/0134616 A1, 2015/0368710 A1, and 2018/0057870 A1, and published International Applications WO 2013/154999, WO2015/148402, WO 2017/042038, and WO 2019/166457 A1, each of which is hereby incorporated herein by reference in its entirety.

As noted in the description above, and elsewhere herein, the incorporation of the tagged nucleotide also results in the extension of a nucleic acid strand. In the context of a polymerase adjacent to a nanopore, and without intending to be bound by mechanism, it is believed that the extended strand can thread into the nearby nanopore and interfere with further detection of tag moieties as the polymerization proceeds. Moreover, it is believed that this template threading phenomenon can result in abbreviated sequence reads and overall lower throughput for nucleic acid detection and/or sequencing using a nanopore. It is an unexpected result and surprising advantage of the present disclosure that the use of primers comprising certain structures, such as blocking moieties, can reduce or prevent the deleterious template threading and greatly improved results in throughput for nucleic acid detection and/or sequencing using a nanopore.

Generally, the threading-blocker primers of the present disclosure comprise a compound of formula (I):

5′-[Blocking Moiety]-[Primer]-3′ (I)

wherein, the Blocking Moiety comprises a poly-cationic group, a bulky group, or a base-modified nucleoside, wherein the base-modified nucleoside comprises a poly-cationic group or a bulky group attached to the nucleoside base; and the Primer comprises an oligonucleotide capable of priming polymerization of the copy strand by a polymerase linked to a nanopore. Exemplary poly-cationic groups, bulky groups, and base-modified nucleosides useful as a Blocking Moiety of a threading-blocking primer of the present disclosure are described further herein including in the Examples.

Additional embodiments of the threading-blocker primer compounds of formula (I) are described by a range of sub-structures and other properties, as disclosed herein below and include the specific embodiments described in the Examples.

It is contemplated that the Blocking Moiety is attached to the 5′-end of the Primer using oligonucleotide synthesis well-known in the art. Such methods allow for formation of a phosphodiester, a phosphorothioate, an H-phosphonate, or methyl phosphonate linkage between the Blocking Moiety to the Primer. Accordingly, in some embodiments, the threading-blocker primers of formula (I) comprise a compound of formula (Ia)

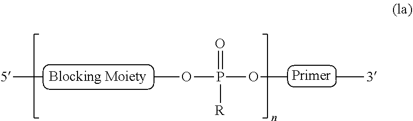

wherein, n is 1 to 10, and R is independently selected from O⁻, S⁻, CH₃, and H. In some embodiments, R is O⁻ and the linkage is a phosphodiester, that is, the Blocking Moiety is phosphodiester-linked to the 5′-end of the Primer.

As described above for the primer compounds of formula (I), of which the compound of formula (Ia) is a sub-structure, the Blocking Moiety of the primer compounds of formula (Ia) can comprise a poly-cationic group, a bulky group, or a base-modified nucleoside, wherein the base-modified nucleoside comprises a poly-cationic group or a bulky group attached to the nucleoside base; and the Primer comprises an oligonucleotide capable of priming polymerization of a copy strand by a polymerase linked to a nanopore. However, as shown by formula (Ia), it is further contemplated that the Blocking Moiety encompasses oligomers of blocking moiety groups, such as polycationic or bulky groups, and such oligomers can comprise phosphodiester, phosphorothioate, H-phosphonate, or methyl phosphonate linkages. As described elsewhere herein, these oligomeric Blocking Moiety can be prepared using commercially available reagents and standard automated oligonucleotide synthesis techniques.

In some embodiments, the threading-blocker primers of formulas (I) or (Ia) comprise a compound of formula (Ib)

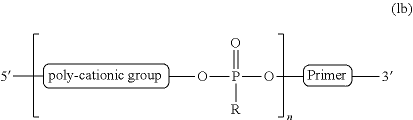

wherein, n is 1 to 10, and R is independently selected from O⁻, S⁻, CH₃, and H.

Exemplary poly-cationic groups are described further herein including in the Examples.

In some embodiments, the threading-blocker primers of formulas (I) or (Ia) comprise a compound of formula (Ic)

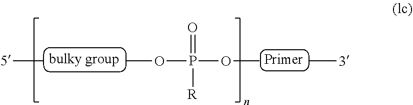

wherein, n is 1 to 10. and R is independently selected from O⁻, S⁻, CH₃, and H. Exemplary bulky groups are described further herein including in the Examples.

As described above, it is contemplated that the Blocking Moiety of the threading-blocker primers of formulas (I) or (Ia) can comprise a base-modified nucleoside, wherein the base-modified nucleoside comprises a poly-cationic group or a bulky group attached to the nucleoside base. In some embodiments, the threading-blocker primers of formula (I) comprise a compound of formula (Id):

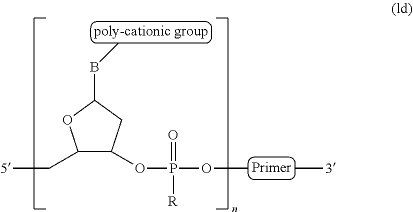

wherein, B is a modified nucleobase, R is independently selected from O⁻, S⁻, CH₃, and H, and n is 1 to 10.

In some embodiments, the threading-blocker primers of formula (I) comprise a compound of formula (Ie):

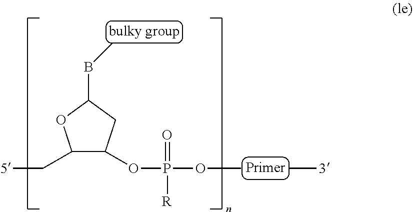

wherein, B is a modified nucleobase, R is independently selected from O⁻, S⁻, CH₃, and H, and n is 1 to 10. In some embodiments of the threading-blocker primers of formula (Id) and (Ie), the Blocking Moiety comprises is not oligomeric and comprises a single base-modified nucleoside. Exemplary base-modified nucleosides are described further herein including in the Examples.

In some embodiments, the present disclosure provides a threading-blocker primer compound of formulas (I), or (Ia)-(Ie), wherein the compound is selected from those listed in Table 1.

TABLE 1
Exemplary threading-blocker primers of formula (I)

| Primer Structure | Blocking Moiety Type |
|---|---|
| 5′-(spermine)₂-[Primer]-3′ | (spermine)₂ |
| | poly-cationic |
| 5′-(spermine)₃-[Primer]-3′ | (spermine)₃ |
| | poly-cationic |
| 5′-(spermine)₄-[Primer]-3′ | (spermine)₄ |
| | Poly-cationic |
| 5′-(spermine)₅-[Primer]-3′ | (spermine)₅ |
| | poly-cationic |
| 5′-(pyrene)₂-[Primer]-3′ | (pyrene)₂ |
| | bulky |
| 5′-(cholestyryl)-[Primer]-3′ | (cholesteryl) |
| | Bulky |
| 5′-(T*-[Phe(4-NO₂)-εLys- | T*-[Phe(4-NO₂)-εLys-(Lys)₁₂] |
| (Lys)₁₂])-[Primer]-3′ | poly-cationic modified |
| | nucleoside |
| 5′-(T*-[(Lys)₁₂-εLys- | T*-[(Lys)₁₂-εLys-Phe(4-NO₂)] |
| Phe(4-NO₂)])-[Primer]-3′ | poly-cationic modified |
| | nucleoside |
| 5′-(T*-[(Lys)₈-εLys- | T*-[(Lys)₈-εLys-Phe(4-NO₂)] |
| Phe(4-NO₂)])-[Primer]-3′ | poly-cationic modified |
| | nucleoside |
| 5′-(T*-[PAMAM Gen1 | T*-[PAMAM Gen1 amino] |
| amino])-[Primer]-3′ | poly-cationic modified |
| | nucleoside |
| 5′-(dU-[perylene])- | dU-[perylene] |
| [Primer]-3′ | bulky group modified |
| | nucleoside |

As described elsewhere herein, the threading-blocker primers of the present disclosure provide the advantage of reducing and/or preventing deleterious threading that can occur during nucleic acid detection and sequencing using a polymerase-linked nanopore device. Such nanopore-based methods can be part of a wide-range of processes well-known in the art that utilize biotin for nucleic acid purification, separation, and isolation. Accordingly, it is contemplated that in some embodiments, the threading-blocker primers of the present disclosure can include a biotin tag that facilitates the purification, separation, and/or isolation of nucleic acid strands incorporating these primers.

In some embodiments, the threading-blocker primers of the present disclosure comprising a compound of formulas (I) (e.g., any of the compounds of formulas (Ia)-(Ie)) can further comprise a biotin tag attached to the 5′-end of the Blocking Moiety.

As used herein, the term “biotin tag” is intended to include a biotin moiety, a desthiobiotin moiety, or an iminobiotin moiety, attached to the 5′-end of the Blocking Moiety directly, or indirectly through a linker moiety. That is, the term “biotin tag” can include a biotin, a desthiobiotin, or an iminobiotin moiety together with a linker moiety.

In some embodiments, the threading-blocker primers of the present disclosure (e.g., compounds of formulas (I), (Ia), and (Ib)-(Ie)) can comprise a compound of formula (II):

5′-[Biotin Tag]-[Blocking Moiety]-[Primer]-3′ (II)

wherein, the Blocking Moiety comprises a poly-cationic group, a bulky group, or a base-modified nucleoside, wherein the base-modified nucleoside comprises a poly-cationic group or a bulky group attached to the nucleoside base; the Biotin Tag comprises a biotin tag; and the Primer comprises an oligonucleotide capable of priming polymerization of a copy strand by a polymerase linked to a nanopore. Exemplary poly-cationic groups, bulky groups, and base-modified nucleosides useful as a Blocking Moiety of a threading-blocking primer of the present disclosure are described further herein including in the Examples. In some embodiments of the compound of formula (II) the Biotin Tag attached to the 5′-end of the Blocking Moiety of the threading-blocker primer of formula (II) can comprise a biotin moiety and a linker moiety, or a desthiobiotin moiety and a linker moiety.

Additional embodiments of the threading-blocker primer compounds of formula (II) are described by a range of sub-structures and other properties, as disclosed herein below and include the specific embodiments described in the Examples.

In some embodiments, the threading-blocker primers of formula (II) comprise a compound of formula (IIa)

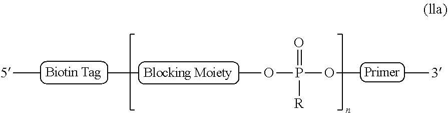

wherein, n is 1 to 10, and R is independently selected from O⁻, S⁻, CH₃, and H. In some embodiments, R is O⁻ and the linkage is a phosphodiester, that is, the Blocking Moiety is phosphodiester-linked to the 5′-end of the Primer.

As described above for the primer compounds of formula (II), of which formula (IIa) is a sub-structure, the Blocking Moiety of the primer compounds of formula (IIa) can comprise a poly-cationic group, a bulky group, or a base-modified nucleoside, wherein the base-modified nucleoside comprises a poly-cationic group or a bulky group attached to the nucleoside base; and the Primer comprises an oligonucleotide capable of priming polymerization of a copy strand by a polymerase linked to a nanopore. However, as shown by formula (IIa), it is further contemplated that the Blocking Moiety encompasses oligomers of blocking moiety groups, such as polycationic or bulky groups, and such oligomers can comprise phosphodiester, phosphorothioate, H-phosphonate, or methyl phosphonate linkages. As described elsewhere herein, these oligomeric Blocking Moiety can be prepared using commercially available reagents and standard automated oligonucleotide synthesis techniques.

In some embodiments, the threading-blocker primers of formula (II) comprise a compound of formula (IIb)

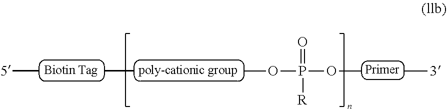

wherein, n is 1 to 10, and R is independently selected from O⁻, S⁻, CH₃, and H. Exemplary poly-cationic groups are described further herein including in the Examples.

In some embodiments, the threading-blocker primers of formula (II) comprise a compound of formula (IIc)

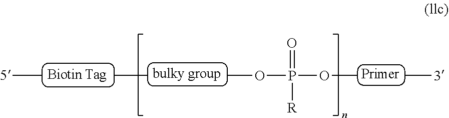

wherein, n is 1 to 10. and R is independently selected from O⁻, S⁻, CH₃, and H. Exemplary bulky groups are described further herein including in the Examples.

As described above, it is contemplated that the Blocking Moiety of the threading-blocker primers of formulas (II) or (IIa) can comprise a base-modified nucleoside, wherein the base-modified nucleoside comprises a poly-cationic group or a bulky group attached to the nucleoside base. In some embodiments, the threading-blocker primers of formula (II) comprise a compound of formula (IId):

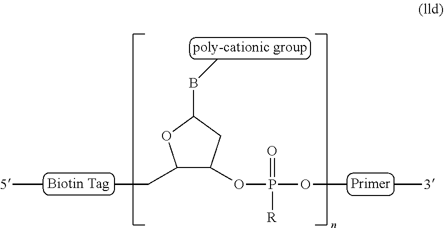

wherein, B is a modified nucleobase, R is independently selected from O⁻, S⁻, CH₃, and H, and n is 1 to 10.

In some embodiments, the threading-blocker primers of formula (II) comprise a compound of formula (IIe):

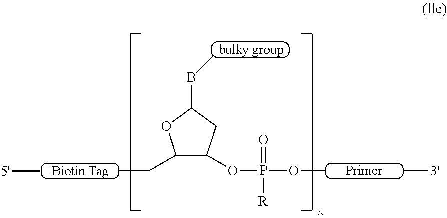

wherein, B is a modified nucleobase, R is independently selected from O⁻, S⁻, CH₃, and H, and n is 1 to 10. In some embodiments of the threading-blocker primers of formula (IId) and (IIe), the Blocking Moiety comprises is not oligomeric and comprises a single base-modified nucleoside. Exemplary base-modified nucleosides are described further herein including in the Examples.

In some embodiments, the present disclosure provides a threading-blocker primer compound of formulas (II), or (IIa)-(IIe), wherein the compound is selected from those listed in Table 2.

TABLE 2
Exemplary threading-blocker primers of formula (I)

| Primer Structure | Blocking Moiety Type |
|---|---|
| 5′-[Biotin Tag]- | (spermine)₂ |
| (spermine)₂-[Primer]-3′ | poly-cationic |
| 5′-[Biotin Tag]- | (spermine)₃ |
| (spermine)₃-[Primer]-3′ | poly-cationic |
| 5′-[Biotin Tag]- | (spermine)₄ |
| (spermine)₄-[Primer]-3′ | Poly-cationic |
| 5′-[Biotin Tag]- | (spermine)₅ |
| (spermine)₅-[Primer]-3′ | poly-cationic |
| 5′-[Biotin Tag]- | (pyrene)₂ |
| (pyrene)₂-[Primer]-3′ | bulky |
| 5′-[Biotin Tag]- | (cholesteryl) |
| (cholestyryl)-[Primer]-3′ | Bulky |
| 5′-[Biotin Tag]-(T*- | T*-[Phe(4-NO₂)-εLys-(Lys)₁₂] |
| [Phe(4-NO₂)-εLys-(Lys)₁₂])- | poly-cationic modified |
| [Primer]-3′ | nucleoside |
| 5′-[Biotin Tag]-(T*- | T*-[(Lys)₁₂-εLys-Phe(4-NO₂)] |
| [(Lys)₁₂-εLys-Phe(4-NO₂)])- | poly-cationic modified |
| [Primer]-3′ | nucleoside |
| 5′-[Biotin Tag]-(T*- | T*-[(Lys)₈-εLys-Phe(4-NO₂)] |
| [(Lys)₈-εLys-Phe(4-NO₂)])- | poly-cationic modified |
| [Primer]-3′ | nucleoside |
| 5′-[Biotin Tag]-(T*- | T*-[PAMAM Gen1 amino] |
| [PAMAM Gen1 amino])- | poly-cationic modified |
| [Primer]-3′ | nucleoside |
| 5′-[Biotin Tag]- | dU-[perylene] |
| (dU-[perylene])-[Primer]-3′ | bulky group modified |
| | nucleoside |

As disclosed elsewhere herein, the addition of a 5′ biotin tag to the threading-blocker primers of the present disclosure can facilitate further processing of the extended nucleic acid strand incorporating the primer, such as purification, separation, and/or isolation, in various well-known nucleic acid processes or assays. It is further contemplated that in some processes it is desirable to cleave the biotin tag from the extended nucleic acid strand, e.g., after strand extension polymerization, in order to facilitate other processes or assays using the nucleic acid.

Accordingly, in some embodiments of the threading-blocker primers of the present disclosure (e.g., compounds of formulas (I) and (II)) the biotin tag attached to the 5′-end of the Blocking Moiety comprises an oligonucleotide of a sequence that is selectively cleavable (e.g., enzymatically cleavable). In some embodiments. the biotin tag comprises an oligonucleotide sequence that is selectively cleavable by an enzyme, such as the sequence, TTTTUUU (SEQ ID NO: 14). Thus, in some embodiments, any of the threading-blocker primer compounds of the present disclosure can comprise a biotin tag attached to the 5′-end of the Blocking Moiety, wherein the biotin tag comprises an oligonucleotide having a sequence selected from: TTTTUUU (SEQ ID NO: 15); TTTTUUUT (SEQ ID NO:16); TTTTUUUTT (SEQ ID NO: 17); TTTUUUTTT (SEQ ID NO: 18); TTTTUUUTTTT (SEQ ID NO: 19); TTTTUUTTTTTUUT (SEQ ID NO: 20); TUUTTTTUU (SEQ ID NO:21); TUUTTTTTTUU (SEQ ID NO: 22); and TTTTUUUUUU (SEQ ID NO: 23).

In any of the embodiments of the threading-blocker primer compounds disclosed herein comprising a biotin tag attached to the 5′-end of the Blocking Moiety (e.g., compounds of formulas (II), and (IIa)-(IIe)), the biotin tag can comprise a structure of formula (III):

B-L-[(N)ₓ—(U)_{y}—(N)_{z}]_{w} (III)

wherein, B is biotin or desthiobiotin; L is a linker; N is a nucleotide; U is uracil; and x and z are at least 1; y is at least 3; and w is 0 or 1.

In any of the embodiments of the threading-blocker primer compounds disclosed herein comprising a biotin tag attached to the 5′-end of the Blocking Moiety (e.g., compounds of formulas (II) and (IIa)), the biotin tag can comprise a structure selected from: 5′-(Biotin)-(Sp18)-TTTUUUTT-3′; 5′-(Desthiobiotin)-(Sp18)-TTTUUUTT-3′; 5′-(BiotinTEG)-(Sp18)₂-TTTUUUTT-3′; 5′-(DesthiobiotinTEG)-(Sp18)₂-TTTUUTT-3′; 5′-(BiotinTEG)-(Sp18)₃-3′; 5′-(DesthiobiotinTEG)-(Sp18)₃-TTTUUUTT-3′; or 5′-(Biotin)₂-(Sp18)-TTTUUUTT-3′.

A wide range of phosphoramidite reagents are available that can be used to prepare and/or attach a biotin tag to the 5′-end of a threading-blocker primer of the present disclosure. For example, the commercially available phosphoramidite reagents (e.g., available from Glen Research, Inc., Sterling, Va., USA) shown below in Table 3 can be used in standard automated oligonucleotide synthesis to attach a biotin moiety or desthiobiotin moiety, either directly or through a spacer or linker (e.g., Sp18), to the 5′-end of a threading-blocker primer.

TABLE 3

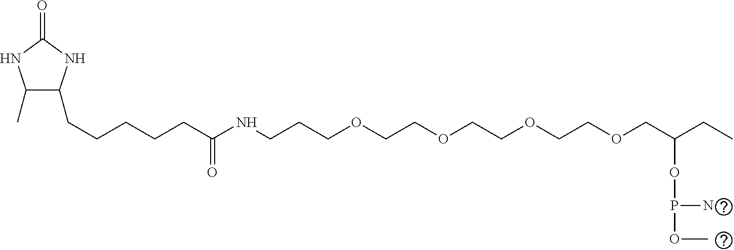 [SMILES: CCC(COCCOCCOCCOCCCNC(=O)CCCCCC1NC(=O)NC1C)OP(N)OC]

“Desthiobiotin-TEG phosphoramidite”
(chemical name: 1-Dimethoxytrityloxy-3-O-(N-desthiobiotinyl-3-aminopropyl)-
triethyleneglycolyl-glyceryl-2-O-(2-cyanoethyl)-(N,N-diisopropyl)-phosphoramidite)

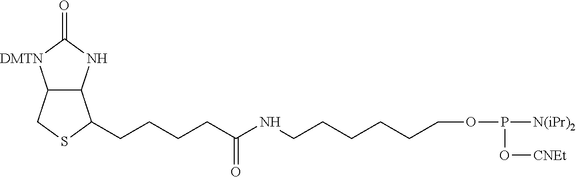

“5′-biotin phosphoramidite”
(chemical name: [1-N-(4,4′-Dimethoxytrityl)-biotinyl-6-aminohexyl]-2-cyanoethyl-(N,N-
diisopropyl)-phosphoramidite)

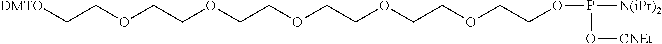

“Spacer phosphoramidite 18”
(chemical name: “18-O-Dimethoxytritylhexaethyleneglycol,1-[(2-cyanoethyl)-(N,N-
diisopropyl)]-phosphoramidite”; forms “Sp18” spacer monomer unit in oligonucleotide)

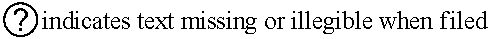

As disclosed else herein, a general structural feature of the threading-blocker primers of the present disclosure (e.g., compounds of formulas (I), (Ia), (II), or (IIa)) include a Blocking Moiety structure attached to the 5′-end of a Primer structure. The Blocking Moiety can comprise a poly-cationic group, a bulky group, or a base-modified nucleoside, wherein the base-modified nucleoside comprises a poly-cationic group or a bulky group attached to the nucleoside base. Without intending to be bound by theory or mechanism, it is believed that the strand-displacing activity of a strand-extending enzyme (e.g., Pol6 DNA polymerase) attached proximal to a nanopore causes threading of the primer extended strand into the nanopore which threading is deleterious to the continued function of the nanopore in detecting the tagged nucleotides being incorporated by the enzyme, and effectively stops the nanopore device from making further “reads” after only a short procession. The presence of a Blocking Moiety attached to the 5′ end of the Primer sequence effectively reduces or prevents this deleterious template threading phenomenon. As noted above, an effective Blocking Moiety can have a range of different structures selected from: (a) a poly-cationic group; (b) a bulky group; or (c) a base-modified nucleoside, wherein the base-modified nucleoside comprises a poly-cationic group or a bulky group attached to the nucleoside base. Various embodiments of the Blocking Moiety useful in the threading-blocker primer compounds of the present disclosure comprise a range of sub-structures and other properties, as disclosed herein below, and can include the specific embodiments described in the Examples.

In some embodiments, the Blocking Moiety comprises a poly-cationic group. Exemplary poly-cationic groups useful as Blocking Moieties in the compounds of the present disclosure can include oligomers of cationic aminoalkyl groups such as spermine, spermidine, ethylenediamine, propylene diamine, allylamine. Accordingly, in some embodiments the Blocking Moiety comprises a poly-cationic group selected from poly(spermine), poly(spermidine), poly(ethylenediamine), poly(propylenediamine), poly(allylamine). In some embodiments, Blocking Moieties useful in the primer compounds of the present disclosure include:oligomers of spermine, including the following oligomers: (spermine)₂, (spermine)₃, (spermine)₄, and (spermine)₅.

Generally, where the Blocking Moiety comprises a poly-cationic group, the group comprises an oligomer of cationic groups (e.g., spermine). However, it is contemplated that in some embodiments, these oligomers of cationic groups can be prepared using standard automated oligonucleotide synthesis that results in phosphodiester-linked oligomers. A wide range of phosphoramidite reagents are available that result in phosphodiester-linked oligomers that are cationic aminoalkyl groups. For example, the reagent, spermine phosphoramidite (shown below) is commercially available (e.g., from Glen Research, Inc.) and can be used in standard automated oligonucleotide synthesis to attach one or more spermine poly-cationic groups onto a threading-blocker primer of the present disclosure.

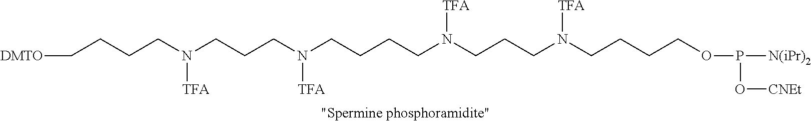

### (chemical name: N¹-[4-(4,4′-Dimethoxytrityloxy)butyl]-N¹,N4,N9,N¹²-tetrakis(trifluoroacetyl)-spermine-N¹²-butyl-4-[(2-cyanoethyl)-(N,N-diisopropyl)]-phosphoramidite)

The one or more spermine cationic groups incorporated into an oligonucleotide using spermine phosphoramidite are linked via a phosphodiester linkage formed in standard phosphoramidite synthesis. Accordingly, in some embodiments, wherein the Blocking Moiety comprises an oligomer of spermine groups, the oligomers are phosphodiester-linked.

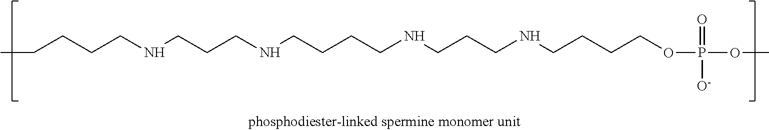

In some embodiments, the Blocking Moiety comprises a poly-cationic group that is an oligomer of a cationic amino acids. Accordingly, in some embodiments the Blocking Moiety comprises an oligomer of cationic amino acids selected from: lysine, ε-lysine, omithine, (aminoethyl)glycine, arginine, histidine, methyllysine, dimethyllysine, trimethyllysine, and/or aminoproline. In some embodiments, Blocking Moieties useful in the primer compounds of the present disclosure based on oligomers of cationic amino acids include: [Phe(4-NO₂)-εLys-(Lys)₈], [Phe(4-NO₂)-εLys-(Lys)₁₂], [(Lys)₈-εLys-Phe(4-NO₂)], [(Lys)₁₂-εLys-Phe(4-NO₂)], [PAMAM Gen1 amino].

In some embodiments, the Blocking Moiety comprises a bulky group. Exemplary bulky groups useful for the primer compounds of the present disclosure include, but are not limited to, an aryl group, an arylalkyl group, a heteroaryl group, a heteroarylalkyl group, a cycloalkyl group, a heterocycloalkyl group, or some combination of any of these bulky groups. In some embodiments, the bulky group can be selected from a pyrene, a cholesteryl, a perylene, a perylenediimide, a cucurbituril, a beta-cyclodextrin, a high poly(ethylene glycol) polymer, or a combination of any of these bulky groups.

In some embodiments, it is contemplated that the Blocking Moiety comprises a bulky group, wherein the bulky group comprises an oligomer of bulky groups, such as a pyrene, a cholesteryl, a perylene, a perylenediimide, a cucurbituril, a beta-cyclodextrin, a high poly(ethylene glycol) polymer (e.g., PEG polymer), or some combination thereof. As described elsewhere herein, a wide range of phosphoramidite reagents are available that result in phosphodiester-linked oligomers that can include oligomers of bulky groups. Accordingly, in some embodiments, wherein the Blocking Moiety comprises an oligomer of bulky groups, the bulky group can be phosphodiester-linked bulky group.

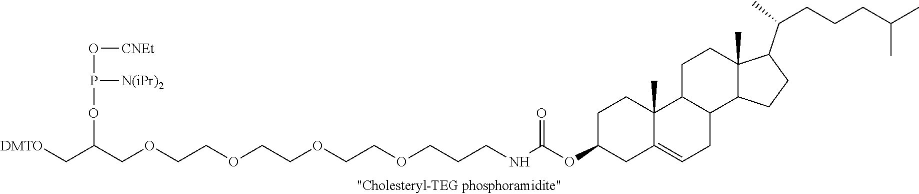

### (chemical name: 1-Dimethoxytrityloxy-3-O—(N-cholesteryl-3-aminopropyl)-triethyleneglycol-glyceryl-2-O-(2-cyanoethyl)-(N,N,-diisopropyl)-phosphoramidite)

The one or more cholesteryl bulky groups can be incorporated into an oligonucleotide using Cholesteryl-TEG phosphoramidite linked via a phosphodiester linkage formed in standard phosphoramidite synthesis. Accordingly, in some embodiments, wherein the Blocking Moiety comprises one or more bulky groups, the oligomers are phosphodiester-linked cholesteryl.

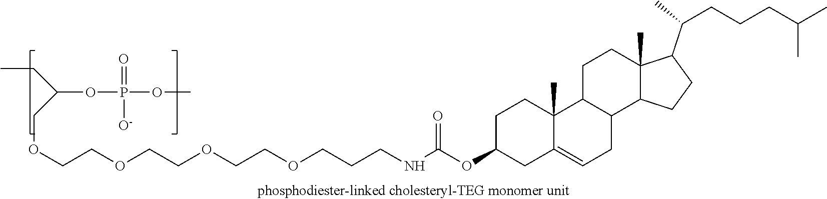

As described elsewhere herein, the Primer of the compounds of the present disclosure (e.g., compound of formula (I)) comprises an oligonucleotide capable of priming polymerization of a copy strand by a polymerase linked to a nanopore. Accordingly, in some embodiments, the Blocking Moiety attached to the 5′-end of the Primer can be an oligomer of phosphodiester-linked groups prepared using standard automated oligonucleotide synthesis that are not nucleosides. For examples, the oligomers of phosphodiester-linked poly-cationic groups or bulky groups. It is also contemplated, however, that the Blocking Moiety can comprise a base-modified nucleoside. Base-modified (or base-modifiable) nucleosides are well-known and can be easily attached to the 5′-end of an oligonucleotide primer using standard automated oligonucleotide synthesis.

Accordingly, in some embodiments of the compounds of the present disclosure, the Blocking Moiety comprises a base-modified nucleoside, wherein the base-modification comprises a poly-cationic group or a bulky group. It is contemplated that any of the poly-cationic groups or bulky groups disclosed herein can also be used in the base-modified nucleoside embodiments. Thus, in some embodiments, the base-modification can comprise a poly-cationic group selected from poly-lysine, poly-arginine, poly-histidine, poly-ornithine, poly-(aminoethyl)glycine, poly-methyllysine, poly-dimethyllysine, poly-trimethyllysine, poly-aminoproline, and poly-ε-lysine. In some embodiments, the base-modification can comprise a bulky group selected from perylene, cholesteryl, and beta-cyclodextrin.

Methods for preparing a base-modified nucleoside are well-known in the art. The copper(I)-catalyzed azide-alkyne cycloaddition (CuAAC) reaction between azides and alkynes can be used to form covalent 1,2,3-triazole linkage to an alkyne-modified nucleobase previously incorporated into an oligonucleotide prepared by standard automated synthesis using phosphoramidite reagents. A variety of phosphoramidite reagents that result in an alkyne-modified nucleobase are commercially available (see e.g., Glen Research, Sterling, Va., USA). Any of these reagents can be used with standard automated oligonucleotide synthesis methods followed CuAAC modification to provide an oligonucleotide with a modified T (or dU) that is base-modified with a poly-cationic or bulky group. Exemplary phosphoramidite reagents useful in preparing threading-blocker primers with base-modified Blocking Moiety are provide in Table 4.

TABLE 4
Phosphoramidite reagents useful for CuAAC base-modification

| Phosphoramidite Reagent Structure | Chemical Name |
|---|---|
| 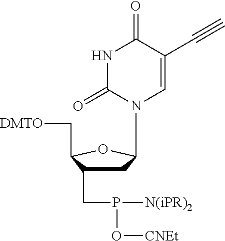 | “5-ethynyl-dU-CE phosphoramidite” (or, “5′-Dimethoxytrityl-5-ethynyl-2′- deoxyUridine, 3′-[(2-cyanoethyl)- (N,N-diisopropyl)]-phosphoramidite”) |
| 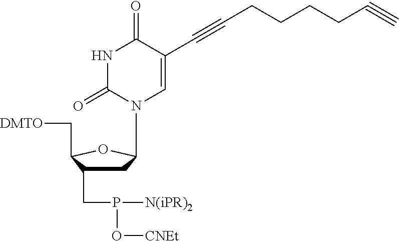 | “C8-alkyne-dT-CE phosphoramidite” (or “5′-Dimethoxytrity-5-(octa-1,7- diynyl)-2′-deoxyuridine, 3′-[(2- cyanoethyl)-(N,N-diisopropyl)]- phosphoramidite”) |

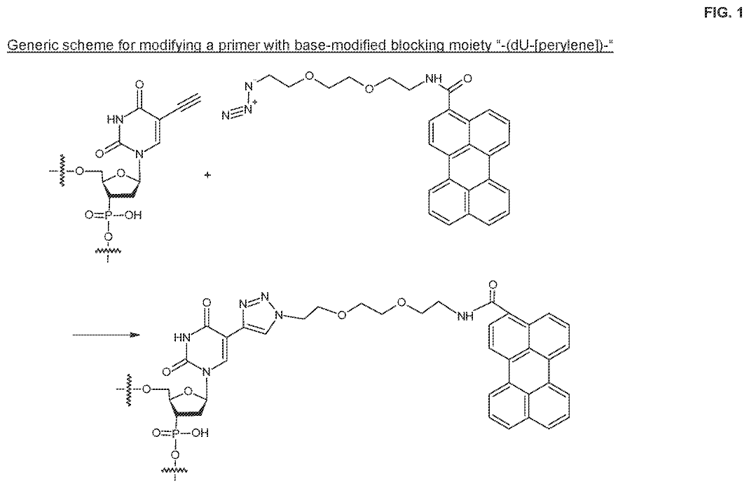

A general example of this type of CuAAC reaction is depicted schematically in FIG. 1. A single 5-ethynyl-dU nucleoside within an oligonucleotide (rest of sequence not shown) is prepared using the 5-ethynyl-dU-CE phosphoramidite reagent. Then an azide-modified perylene compound is reacted with this alkyne-modified oligonucleotide under standard CuAAC reaction conditions to result in an oligonucleotide that includes a single nucleobase modified with a perylene bulky group. This type of modification which results in a short linker to the nucleobase is indicated in an oligonucleotide sequence formula as a “-(dU-[perylene])-” monomer unit.

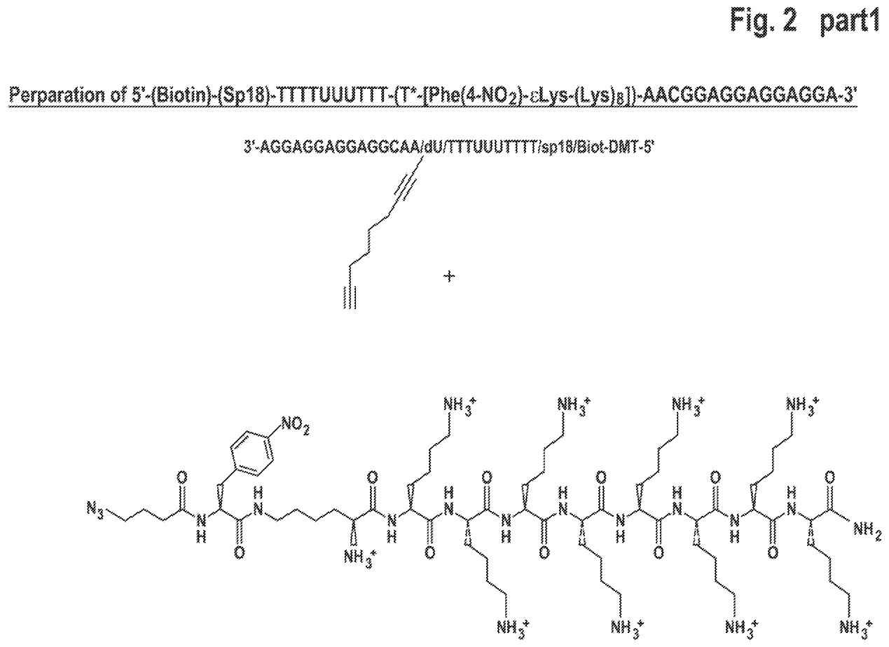

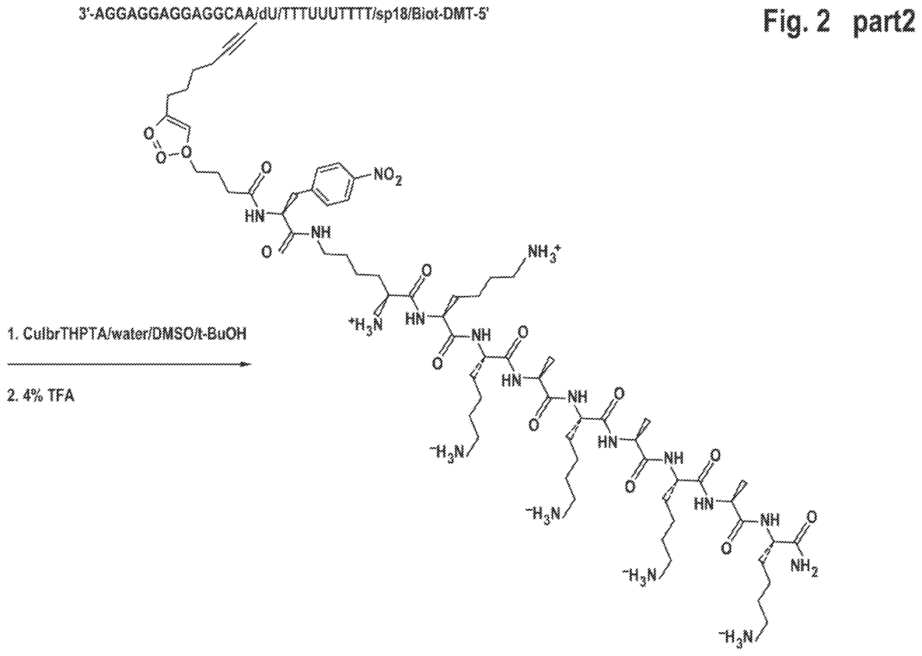

An exemplary CuAAC reaction for preparing a threading-blocker primer of the present disclosure is depicted in FIG. 2. The starting oligonucleotide, 5′-(Biotin)-(Sp18)-TTTTUUUTTT-(C8-alkyne-dT)-AACGGAGGAGGAGGA-3′, is prepared via standard oligonucleotide synthesis using the reagent, C8-alkyne-dT-CE phosphoramidite, to insert the C8-alkyne modified dT unit (also referred to herein as “T*”). Then, the azide-modified 8-lysine polypeptide, Phe(4-NO₂)-εLys-(Lys)₈, is reacted with the alkyne-modified oligonucleotide under standard CuAAC reaction conditions. The resulting product is the threading-blocker primer, 5′-(Biotin)-(Sp18)-TTTTUUUTTT-(T*-[Phe(4-NO₂)-εLys-(Lys)])-AACGGAGGAGGAGGA-3′. The exemplary Blocking Moiety comprises the T nucleoside base-modified with an 8 carbon linker covalently attached through a triazole group to an 8 lysine poly-cationic group, [Phe(4-NO₂)-εLys-(Lys)₈].

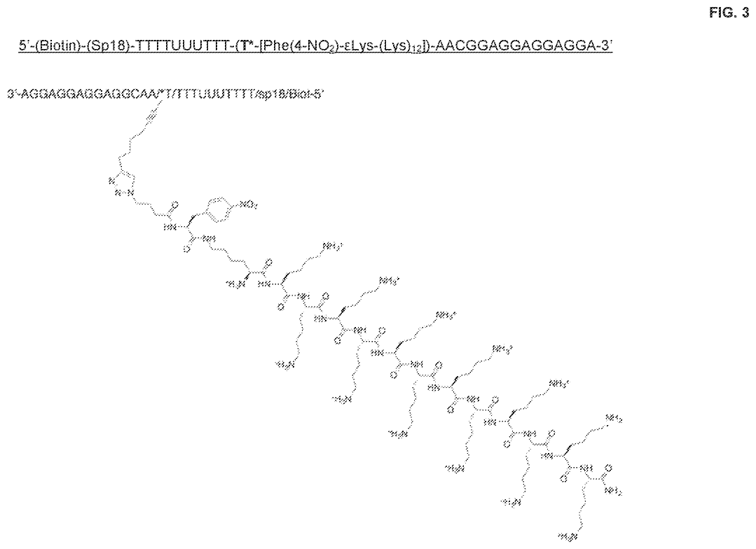

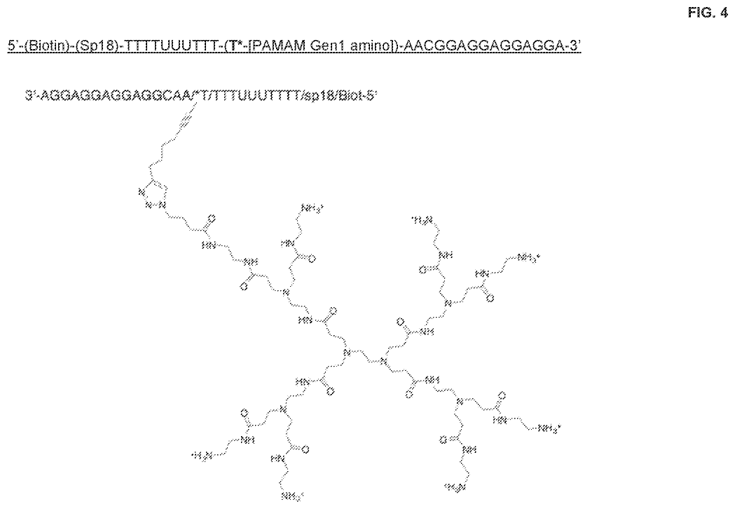

FIGS. 3 and 4 depict exemplary threading-blocker primer compounds comprising a Blocking Moiety, wherein the Blocking Moiety comprises a base-modified nucleoside, wherein the base modification is a poly-cationic group. FIG. 3 depicts a primer compound, 5′-(Biotin)-(Sp18)-TTTUUUTT-(T*-[Phe(4-NO₂)-(ε-Lys)-(Lys)₁₂])-TAACGGAGGAGGAGGA-3′. This compound features Blocking Moiety comprising a T* nucleoside, which is a T nucleoside that is base-modified at the 5-position with a C8 linker (e.g., using “C8-alkyne-dT-CE phosphoramidite”) and then further linked through a CuAAC-formed triazole to the poly-cationic group [Phe(4-NO₂)-(ε-Lys)-(Lys)₁₂]. FIG. 3 depicts the primer compound, 5′-(Biotin)-(Sp18)-TTTUUUTT-(T*-[PAMAM Gen1 amino])-TAACGGAGGAGGAGGA-3′. This compound features a Blocking Moiety also comprising a T nucleoside that is base-modified via a triazole linkage to the “PAMAM Gen1 amino” poly-cationic group, which has a dendritic structure comprising seven positively-charged amine groups as shown in the FIG. 3.

In some embodiments, the present disclosure provides a threading-blocker primer compound of formulas (I) or (II), wherein the compound is selected from those exemplary compounds listed in Table 5.

TABLE 5

| Threading Blocker Primer | SEQ ID NO: |
|---|---|
| 5′-(Biotin)-(Sp18)-TTTTUUUTT-(spermine)-TAACGGAGGAGGAGGA-3′ | 24 |
| 5′-(Biotin)-(Sp18)-TTTUUUTT-(spermine)₂-TAACGGAGGAGGAGGA-3′ | 25 |
| 5′-(Biotin)-(Sp18)-TTTUUUTT-(spermine)₃-TAACGGAGGAGGAGGA-3 | 26 |
| 5′-(Biotin)-(Sp18)-TTTUUUTT-(spermine)₄-TAACGGAGGAGGAGGA-3′ | 27 |
| 5′-(Biotin)-(Sp18)-TTTUUUTT-(spermine)₅-TAACGGAGGAGGAGGA-3′ | 28 |
| 5′-(Biotin)-(Sp18)-TTTTUUUTT-(spermine)₅-TAACGGAGGAGGAGGA-3′ | 29 |
| 5′-(Biotin)-(Sp18)-TUUTTTTUU-(spermine)₃-TAACGGAGGAGGAGGA-3′ | 30 |
| 5′-(Biotin)-(Sp18)-TUUTTTTTUU-(spermine)₂-TAACGGAGGAGGAGGA-3′ | 31 |
| 5′-(Biotin)-(Sp18)-TTTTUUUUUU-(spermine)₂-TAACGGAGGAGGAGGA-3′ | 32 |
| 5′-(Biotin)-(Sp18)-TTTTUUUTTT-(ethylendiamine)₅-TAACGGAGGAGGAGGA-3′ | 33 |
| 5′-(Biotin)-(Sp18)-TTTTUUUTTT-(ethylendiamine)₆-TAACGGAGGAGGAGGA-3′ | 34 |
| 5′-(Biotin)-(Sp18)-TTTUUUTT-(pyrene)₂-TAACGGAGGAGGAGGA-3′ | 35 |
| 5′-(Biotin)-(Sp18)-TTTUUUTT-(cholesteryl)-TAACGGAGGAGGAGGA-3′ | 36 |
| 5′-Biotin-(Sp18)-TTTTUUUTTT-(T*[Phe(4-NO₂)-(ϵ-Lys)-(Lys)₈])-AACGGAGG | 37 |
| AGGAGGA-3′ | |
| 5′-(Biotin)-(Sp18)-TTTUUUTT-(T*[Phe(4-NO₂)-(ϵ-Lys)-(Lys)₁₂)-TAACGGAG | 38 |
| GAGGAGGA-3′ | |
| 5′-(Biotin)-(Sp18)-TTTUUUTT-(T*PAMAM Gen1 amino))-TAACGGAGGAGGAGG | 39 |
| A-3′ | |
| 5′-(Biotin)-(Sp18)-TTTUUUTT-(dU-[perylene])-TAACGGAGGAGGAGGA-3 | 40 |
| 5′-(Biotin)2-(Sp18)-TTTUUUTT-(dU-[perylene])-TAACGGAGGAGGAGGA-3′ | 41 |
| 5′-(Biotin)-(Sp18)-TTTTUUUTTT-(T*[Phe(4-NO₂)-(ϵ-Lys)-(Lys)₈]-AACGGAG | 42 |
| GAGGA*G*G*A-3′ | |
| 5′-(Biotin)-(Sp18)-TTTTUUUTTT-(T*-[dbco-Phe(4-NO₂)-(ϵ-Lys)-(Lys)₈])- | 43 |
| AACGGAGGAGGA*G*G*A-3′ | |
| 5′-(DesthioblotinTEG)-(Sp18)2-(T*[Phe(4-NO₂)-(ϵ-Lys)-(Lys)₈])- | 44 |
| AACGGAGGAGGAGGA-3′ | |
| 5′-(DesthiobiotinTEG)-(S18)3-(T*[Phe(4-NO₂)-(ϵ-Lys)-(Lys)₈])- | 45 |
| AACGGAGGAGGAGGA-3′ | |
| 5′-(DesthiobiotinTEG)-(Sp18)a-(T*-[Phe(4-NO₂)-(ϵ-Lys)-(Lys)₁₂])-AACG | 46 |
| GAGGAGGAGGA-3′ | |
| 5′-(DesthiobiotinTEG)-(Sp18)3-(spermine)₂-TAACGGAGGAGGAGGA-3′ | 47 |
| 5′-(DesthiobiotinTEG)-(Sp18)3-(spermine)₃-TAACGGAGGAGGAGGA-3′ | 48 |
| 5′-(DesthiobiotinTEG)-(Sp18)3-(spermine)₄-TAACGGAGGAGGAGGA-3′ | 49 |
| 5′-(DesthiobiotinTEG)-(Sp18)3-(spermine)₅-TAACGGAGGAGGAGGA-3′ | 50 |
| 5′-(Biotin)-(Sp18)-TTTTUUUT-(spermine)₂-TAA\hat{ }CGGA\hat{ }GGA\hat{ }GGA\hat{ }GGA-3′ | 51 |
| 5′-(Biotin)-(Sp18)-TTTTUUUT-(spermine)₂-TAA\hat{ }CGGA\hat{ }GGA\hat{ }GGA\hat{ }GGA-3′ | 52 |

As noted elsewhere herein:
“dU-” indicates base-modification at a monomer unit of the oligonucleotide generated using
the 5′-ethynyl-dU-CE phosphoramidite reagent
“T*-” indicates base-modification at a monomer unit of the oligonucleotide generate using
the C8-alkyne-dT-CE phosphoramidinte reagent

Generally, the Primer moiety useful in the threading-blocker primers of the present disclosure can include any primer which is capable of acting as a point of initiation of template-dependent nucleic acid synthesis by a polymerase under conditions suitable for synthesis of a primer extension product complementary to the template strand (i.e., copy strand). In some embodiments, the Primer moiety of the threading-blocker primers of formulas (I) or (II) comprise an oligonucleotide of at least 9-mer, at least 12-mer, or at least 15-mer. In some embodiments, the Primer moiety comprises an oligonucleotide comprising naturally-occurring nucleobase and sugar moieties and phosphodiester linkages between the monomer units. For example, in at least one embodiment the Primer moiety is an oligonucleotide comprising a sequence selected from AACGGAGGAGGAGGA (SEQ ID NO: 53), or AACGGAGGAGGAGGACGTA (SEQ ID NO: 54).

It also contemplated that in some embodiments, the Primer moiety can comprise non-naturally occurring nucleobases and/or sugar moieties. For example, the oligonucleotide can comprise one or more locked nucleic acid units (e.g., a nucleoside unit with a 2′-4′ linkage that “locks” the ribose conformation). In some embodiments, the Primer moiety oligonucleotide comprises a linkage selected from a phosphorothioate, a methyl phosphonate, a phosphotriester, a phosphoramide, and a boronophosphate.

In at least one embodiment, the Primer moiety is an oligonucleotide, wherein the oligonucleotide comprises a one or more locked nucleic acid units; optionally, wherein the oligonucleotide comprises the sequence 5′-TAA\hat{ }CGGA\hat{ }GGA\hat{ }GGA\hat{ }GGA-3′ (SEQ ID NO:55) (wherein A\hat{ } indicates that the A nucleoside is a locked nucleic acid unit).

In at least one embodiment, the Primer moiety is an oligonucleotide, wherein the oligonucleotide comprises a subsequence of phosphorothioate linked nucleoside units at the 3′-end; optionally, wherein the oligonucleotide comprises the sequence 5′-AACGGAGGAGGA*G*G*A-3′ (SEQ ID NO: 56) (wherein * indicates a phosphorothioate linkage).

As noted elsewhere herein, the abbreviations for the modified nucleobases and 3′-capping units are those commonly used for automated oligonucleotide synthesis using commercially available amidite reagents (see e.g., amidite reagent catalogs available from: Glen Research, 22825 Davis Drive, Sterling, Va., USA; or ChemGenes Corp., 33 Industrial Way. Wilmington, Mass., USA). Thus, “SpC2” refers to an abasic 2 carbon spacer; “SpC3” refers to an abasic 3 carbon spacer; “dSp” refers to an abasic ribose spacer, “C3” refers to a 3′-propanol; “N3CEdT” refers to the modified nucleobase that results from the 3-N-cyanoethyl-dT amidite (dT with a cyanoethyl group at position N3); “N3MedT” refers to the modified nucleobase that results from the 3-N-methyl-dT amidite (dT with a methyl group at position N3); “5MedC-PhEt” refers to the modified nucleobase that results from the N4-phenylethyl-5-methyl-dC amidite (5-methyl-dC with phenylethyl at position 4 amine); “Etheno-dA” refers to the modified nucleobase that results from the 1,N6-etheno-dA amidite (dA with ethylene linking N1 to amine position 6); “dCb” refers to modified nucleobase that results from the N4-(O-Levulinyl-6-oxyhexyl)-5-methyl-dC amidite (5-methyl-dC with O-levulinyl-6-oxyhexyl “brancher” at position 4 amine); “Tmp” refers to a thymidine with methylphosphonate linkage; and “Imp” refers to an inosine with methylphosphonate linkage.

B. Uses of Threading-Blocker Primers

The threading-blocker primer compounds of the present disclosure are useful nanopore detection and/or sequencing methods wherein a nanopore device is used to detect the tag of a tagged nucleotide as the nucleotide portion is incorporated (or after it is incorporated and released) by a strand-extending enzyme (e.g., polymerase, ligase) located proximal to the nanopore. Although the threading-blocker primers of the present disclosure are exemplified in use with nanopore-polymerase conjugates and tagged nucleotide compounds for nanopore based sequencing-by-synthesis (SBS) methods, it is contemplated that the threading-blocker primers disclosed herein can be used in any method that requires primer extension of a target sequence by a strand extending enzyme located adjacent to a nanopore, and particularly a wide-pore nanopore. As described elsewhere herein, it has been observed that strand-displacing activity of the strand-extending enzyme can cause threading into the nanopore of the extended primer or complementary strand of the target sequence. This threading into the nanopore is deleterious to the function of the nanopore device because it interferes with detection of the tagged nucleotides used in the method and thus, can effectively stop the nanopore device from detecting a sequence after only a short procession.

As illustrated in the Examples herein, the threading-blocker primers of the present disclosure have improved characteristics for reproducible detection by nanopore devices, particularly where wide-pore mutants are employed, and result in reduced deleterious threading and much longer sequence reads than the corresponding primer compounds without a threading-blocker moieties. For example, in some embodiments, a threading-blocker primer of the present disclosure (e.g., compounds of formulas (I) or (II)) is capable of priming polymerization of a copy strand by a polymerase linked to a nanopore with a read length of at least 1000 bp, at least 1500 bp, at least 2000 bp, at least 2500 bp, or more. Also, in some embodiments, a threading-blocker primer of the present disclosure (e.g., compounds of formulas (I) or (II)) is capable of priming polymerization of a copy strand by a polymerase linked to a nanopore with a template threading rate of less than 50%, less than 40%, less than 30%, less than 20%, less than 10%, or less.

Generally, methods, materials, devices, and systems useful for carrying out nanopore-based detection and/or sequencing using the threading-blocker primer compounds of the present disclosure are described in US Pat. Publ. Nos. 2013/0244340 A1, 2013/0264207 A1, 2014/0134616 A1, 2015/0119259 A1, 2015/0368710 A1, and 2018/0057870 A1, and published International Application WO 2019/166457 A1, each of which is hereby incorporated by reference herein.

In at least one embodiment, the present disclosure provides a method for determining the sequence of a nucleic acid comprising: (a) providing a nanopore sequencing composition comprising: a membrane, an electrode on the cis side and the trans side of the membrane, a nanopore with its pore extending through the membrane, an electrolyte solution in contact with both electrodes, an active polymerase situated adjacent to the nanopore, and a primer strand complexed with the polymerase; (b) contacting the nanopore sequencing composition with (i) a strand of the nucleic acid; and (ii) a set of compounds each comprising a different nucleoside-5′-oligophosphate moiety covalently linked to a tag, wherein each member of the set of compounds has a different tag which results in a different flow of ions through a nanopore when the tag enters the nanopore, and at least one of the different tags comprises a negatively-charged polymer moiety which upon entering a nanopore in the presence of ions results in an altered flow of the ions through the nanopore; and (c) detecting the different flows of ions resulting from the entry of the different tags in the nanopore over time and correlating to each of the different compounds incorporated by the polymerase which are complementary to the nucleic acid sequence, and thereby determining the nucleic acid sequence.

In some embodiments, the present disclosure provides a method for determining the sequence of a nucleic acid comprising: (a) providing a nanopore sequencing composition comprising: a membrane, an electrode on the cis side and the trans side of the membrane, a nanopore with its pore extending through the membrane, an electrolyte solution in contact with both electrodes, an active polymerase situated adjacent to the nanopore, and a primer strand complexed with the polymerase; (b) contacting the nanopore sequencing composition with (i) a strand of the nucleic acid; and (ii) a set of tagged nucleotides each with a different tag, wherein each different tag causes a different tag current level across the electrodes when it is situated in the nanopore, and the set comprises at least one compound for wide-pore nanopore detection comprising a negatively-charged polymer moiety of formula (I), as described elsewhere herein.

1. Nanopores

Nanopores, devices comprising nanopores, and methods for making and using them in nanopore detection applications, such as nanopore sequencing using threading-blocker primers of the present disclosure, are known in the art (See e.g., U.S. Pat. Nos. 7,005,264 B2; 7,846,738; 6,617,113; 6,746,594; 6,673,615; 6,627,067; 6,464,842; 6,362,002; 6,267,872; 6,015,714; 5,795,782; and U.S. Publication Nos. 2015/0119259, 2014/0134616, 2013/0264207, 2013/0244340, 2004/0121525, and 2003/0104428, each of which are hereby incorporated by reference in their entirety). Nanopores, and nanopore devices useful for measuring nanopore detection are also described in the Examples disclosed herein. Generally, the nanopore devices comprise a nanopore embedded in a lipid-bilayer membrane, wherein the membrane is immobilized or attached to a solid substrate which comprises a well or reservoir. The pore of the nanopore extends through the membrane creating a fluidic connection between the cis and trans sides of the membrane. Typically, the solid substrate comprises a material selected from the group consisting of polymer, glass, silicon, and a combination thereof. Additionally, the solid substrate comprises adjacent to the nanopore, a sensor, a sensing circuit, or an electrode coupled to a sensing circuit, optionally, a complementary metal-oxide semiconductor (CMOS), or field effect transistor (FET) circuit. Typically, there are electrodes on the cis and trans sides of the membrane that allow for a DC or AC voltage potential to be set across the membrane which generates a baseline current flow (or O.C. current level) through the pore of the nanopore. The presence of an ion flow altering tag in the nanopore results in change in positive ion flow through the nanopore and thereby generates a measurable change in current level across the electrodes relative to the O.C. current of the nanopore.

It is contemplated that the compositions and methods comprising threading-blocker primers of the present disclosure can be used with a wide range nanopore devices comprising nanopores generated by both naturally-occurring, and non-naturally occurring (e.g., engineered or recombinant) pore-forming proteins. Representative pore forming proteins useful with the compositions and methods include, but are not limited to, α-hemolysin, β-hemolysin, γ-hemolysin, aerolysin, cytolysin, leukocidin, melittin, MspA porin and porin A.

In some embodiments, the nanopore can be formed using the pore-forming protein, α-hemolysin from Staphylococcus aureus (also referred to herein as “α-HL”). α-HL is one of the most-studied members of the class of pore-forming proteins and has been used extensively as the nanopore in nanopore devices. (See e.g., U.S. Publication Nos. 2015/0119259, 2014/0134616, 2013/0264207, and 2013/0244340.) α-HL also has been sequenced, cloned, extensively characterized structurally and functionally using a wide range of techniques including site-directed mutagenesis and chemical labelling (see e.g., Valeva et al. (2001), and references cited therein). The amino acid sequence of the naturally occurring (i.e., wild type) α-HL pore forming protein subunit shown below.

| Wild-Type α-HL amino acid sequence | |
|---|---|
| (SEQ ID NO: 57) | |
| ADSDINIKTG TTDIGSNTTV KTGDLVTYDK ENGMHKKVFY SFIDDKNHNK KLLVIRTKGT | 60 |
| IAGQYRVYSE EGANKSGLAW PASFKVQLQL PDNEVAQISD YYPRNSIDTK EYMSTLTYGF | 120 |
| NGNVTGDDTG KIGGLIGANV SIGHTLKYVQ PDFKTILESP TDKKVGWKVI FNNMVNQNWG | 180 |
| PYDRDSWNPV YGNQLFMKTR NGSMKAADNF LDPNKASSLL SSGFSPDFAT VITMDRKASK | 240 |
| QQTNIDVIYE RVRDDYQLHW TSTNWKGTNT KDKWTDRSSE RYKIDWEKEE MTNGLSAWSH | 300 |
| PQFEK | 305 |

The wild-type α-HL amino acid sequence of SEQ ID NO: 57 does not include the initial methionine residue typically present upon cloning in E. coli and is used for identification of the sequence positions of α-HL amino acid substitutions.

A variety of non-naturally occurring α-HL pore forming proteins have been made including, without limitation, variant α-HL subunits comprising one or more of the following substitutions: H35G, E70K, H144A, E111N, M113A, D127G, D128G, D128K, T129G, K131G, K147N, and V149K. Properties of these various engineered α-HL pore polypeptides are described in e.g., U.S. Published Patent Application Nos. 2017/0088588, 2017/0088890, 2017/0306397, and 2018/0002750, each of which is hereby incorporated by reference herein.

2. Wide-Pore Mutant α-HL Nanopores

It is contemplated that the compositions and methods comprising threading-blocker primers described herein can be used with nanopore devices having wide-pore mutants of α-HL. The wide-pore mutants are non-naturally occurring α-HL proteins that are engineered to form a heptameric nanopore having a constriction site of about 13 angstroms diameter located at a depth of about 65 angstroms as measured from the widest portion of the cis side of the pore when it is embedded in a membrane. In some embodiments, the wide-pore mutants comprise α-HL subunits comprising at least amino acid substitutions E111N and M113A. In some embodiments, the wide-pore mutants comprise α-HL subunits comprising the amino acid substitutions E111N and M113A, and further comprising one or more amino acid substitutions selected from D127G, D128G, D128K, T129G, K131G, K147N, and V149K. The 6:1 heptameric subunit compositions of exemplary wide-pore mutants useful with the compounds, compositions, and methods of the present are disclosed below in Table 6.

TABLE 6

| Mutant | Amino Acid Substitutions |
|---|---|
| Identifier | (positions relative to wild-type α-HL of SEQ ID NO: 57_) |
| P-01 | 6x subunits¹: (H35G, E111N, M113A, D127G, D128G, T129G, |
| | K131G, H144A, K147N, V149K) |
| | 1x subunit²: (E111N, M113A, D127G, D128G, T129G, K131G, |
| | K147N) + C-terminal SpyTag peptide fusion |
| P-02 | 6x subunits: (A1K, S3K, H35G, E111N, M113A, D127G, D128G, |
| | T129G, K131G, H144A, K147N, V149K) |
| | 1x subunit: (H35G, E111N, M113A, D127G, D128G, T129G, K131G, |
| | K147N) + C-terminal SpyTag peptide fusion |
| P-03 | 6x subunits: (0K, A1K, H35G, E111N, M113A, D127G, D128G, |
| | T129G, K131G, H144A, K147N, V149K) |
| | 1x subunit: wild-type + C-terminal SpyTag peptide fusion |
| P-04 | 6x subunits: (H35G, E111N, M113A, D127G, D128G, T129G, |
| | K131G, H144A, K147N, V149K) |
| | 1x subunit: (H35G, E111N, M113A, D127G, D128G, T129G, K131G, |
| | K147N) + C-terminal SpyTag peptide fusion |
| P-05 | 6x subunits: (H35G, E111N, M113A, D127G, D128K, T129G, |
| | K131G, H144A, K147N, V149K) |
| | 1x subunit: (H35G, E111N, M113A, D127G, D128G, T129G, K131G, |
| | K147N) + C-terminal SpyTag peptide fusion |
| P-06 | 6x subunits: (D2N, H35G, S106K, E111N, M113A, D127G, D128G, |
| | T129G, K131G, H144A, K147N, V149K) |
| | 1x subunit: (H35G, E111N, M113A, D127G, D128G, T129G, K131G, |
| | K147N) + C-terminal SpyTag peptide fusion |
| P-07 | 6x subunits: (H35G, E111N, M113A, D127G, D128K, T129G, |
| | K131G, H144A, K147N, V149K, M298A, Ss07d) |
| | 1x subunit: (H35G, E111N, M113A, D127G, D128G, T129G, K131G, |
| | K147N) + C-terminal SpyTag peptide fusion |
| P-08 | 6x subunits: (H35G, E70K, E111N, M113A, D127G, D128K, T129G, |
| | K131G, H144A, K147N, V149K) |
| | 1x subunit: (H35G, E111N, M113A, D127G, D128G, T129G, K131G, |
| | K147N) + C-terminal SpyTag peptide fusion |
| P-09 | 6x subunits: (H35G, N47K, E111N, M113A, D127G, D128K, T129G, |
| | K131G, H144A, K147N, V149K) |
| | 1x subunit: (H35G, E111N, M113A, D127G, D128G, T129G, K131G, |
| | K147N) + C-terminal SpyTag peptide fusion |
| P-10 | 6x subunits: (D13K, H35G, E111N, M113A, D127G, D128K, T129G, |
| | K131G, H144A, K147N, V149K) |
| | 1x subunit: (H35G, E111N, M113A, D127G, D128G, T129G, K131G, |
| | K147N) + C-terminal SpyTag peptide fusion |
| P-11 | 6x subunits: (D13H, H35G, E111N, M113A, D127G, D128K, T129G, |
| | K131G, H144A, K147N, V149K) |
| | 1x subunit: (H35G, E111N, M113A, D127G, D128G, T129G, K131G, |
| | K147N) + C-terminal SpyTag peptide fusion |
| P-12 | 6x subunits: (N17K, H35G, E111N, M113A, D127G, D128K, T129G, |
| | K131G, H144A, K147N, V149K) |
| | 1x subunit: (H35G, E111N, M113A, D127G, D128G, T129G, K131G, |
| | K147N) + C-terminal SpyTag peptide fusion |

¹The “6x subunits” correspond to the α-HL subunits, truncated at position N293, that constitute 6 monomers of the heptameric α-HL nanopore complex.
²The “1x subunit” corresponds to the α-HL subunit that constitutes the 1 monomer modified with a C-terminal SpyTag peptide fusion sequence (e.g., at position N293) that allows conjugation to a SpyCatcher protein-modified strand-extending enzyme, such as Pol6.

As noted in Table 6, in some embodiments, the wide-pore mutant subunits of α-HL can also be truncated at amino acid N293. Additionally, the wide-pore mutants can further include a C-terminal SpyTag peptide fusion and/or His tag as disclosed in WO2017/125565A1, which is hereby incorporated by reference herein, and is further described below. The amino acid sequence of the α-HL pore forming protein subunit truncated at position N293 shown below.

| α-HL amino add sequence subunit truncated at N293 | |
|---|---|
| (SEQ ID NO: 58) | |
| ADSDINIKTG TTDIGSNTTV KTGDLVTYDK ENGMHKKVFY SFIDDKNHNK KLLVIRTKGT | 60 |
| IAGQYRVYSE EGANKSGLAW PSAFKVQLQL PDNEVAQISD YYPRNSIDTK EYMSTLTYGF | 120 |
| NGNVTGDDTG KIGGLIGANV SIGHTLKYVQ PDFKTILESP TDKKVGWKVI FNNMVNQNWG | 180 |
| PYDRDSWNPV YGNQLFMKTR NGSMKAADNF LDPNKASSLL SSGFSPDFAT VITMDRKASK | 240 |
| QQTNIDVIYE RVRDDYQLHW TSTNWKGTNT KDKWTDRSSE RYKIDWEKEE MTN | 293 |

3. Conjugation to Nanopores

It is well-known that a heptameric complex of α-HL monomers spontaneously forms a nanopore that embeds in and creates a pore through a lipid bilayer membrane. It has been shown that heptamers of α-HL comprising a ratio of 6:1 native α-HL subunit to mutant α-HL subunit can form nanopores (see e.g., Valeva et al. (2001) “Membrane insertion of the heptameric staphylococcal alpha-toxin pore—A domino-like structural transition that is allosterically modulated by the target cell membrane,” J. Biol. Chem. 276(18): 14835-14841, and references cited therein). One α-HL monomer subunit (i.e., “the 1× subunit”) of the heptameric pore can be covalently conjugated with a DNA-polymerase using a SpyCatcher/SpyTag conjugation method as described in WO 2015/148402 and WO2017/125565A1, each of which is hereby incorporated by reference herein (see also, Zakeri and Howarth (2010), J. Am. Chem. Soc. 132:4526-7). Briefly, a SpyTag peptide is attached as a recombinant fusion to the C-terminus of the 1× subunit of α-HL, and a SpyCatcher protein fragment is attached as a recombinant fusion to the N-terminus of the strand-extending enzyme, e.g., Pol6 DNA polymerase. The SpyTag peptide and the SpyCatcher protein fragment undergo a reaction between a lysine residue of the SpyCatcher protein and an aspartic acid residue of the SpyTag peptide that results in a covalent linkage conjugating the two the α-HL subunit to the enzyme.

Generally, the wide-pore mutant α-HL subunits are used to prepare heptameric α-HL nanopores with the same methods used with wild-type or other engineered α-HL proteins known in the art. Accordingly, in some embodiments, the threading-blocker primer compounds of the present disclosure can be used with a nanopore device, wherein the nanopore is a wide-pore mutant. As shown by the exemplary wide-pore mutants of Table 6, the 6:1 heptameric α-HL wide-pore nanopore has six subunits (i.e., the “6× subunits”) each having the set of mutations as disclosed in Table 6, and one 1× subunit, which has a slightly different set of mutations as shown in Table 6 (e.g., does not include H144A).

In some embodiments, the 6× subunits are engineered to include a C-terminal fusion comprising the 64 amino acid DNA binding protein 7d of Sulfolobus solfataricus (or “Ss07d”), the sequence of which is described at UniProt entry P39476 (see e.g., at www.uniprot.org/uniprot/P39476; sequence version 2, published Jan. 23, 2007). The Ss07d fusion can act to stabilize the polymerase-template complex of a nearby polymerase for increased processivity.

To facilitate conjugation of a DNA polymerase, the 1× subunit includes a C-terminal fusion (beginning at position 293 or 294 of the truncated wild-type sequence) that includes a SpyTag peptide, e.g., AHIVMVDAYK (SEQ ID NO: 59). The SpyTag peptide allows conjugation of the nanopore to a SpyCatcher-modified strand-extending enzyme, such as a Pol6 DNA polymerase. In some embodiments, the C-terminal SpyTag peptide fusion of the wide-pore mutants comprises a linker peptide, e.g., GGSSGGSSGG (SEQ ID NO: 60), a SpyTag peptide, e.g., AHIVMVDAYKPTK (SEQ ID NO: 61), and a terminal His tag, e.g., KGHHHHHH (SEQ ID NO: 62). Thus, the C-terminal SpyTag peptide fusion that comprises the amino acid sequence: GGSSGGSSGGAHIVMVDAYKPTKKGHHHHHH (SEQ ID NO: 63). In some embodiments (e.g., those disclosed in Table 6), the C-terminal SpyTag peptide fusion of SEQ ID NO: 57 is attached at position N293 of the 1× subunit which is truncated relative to the wild-type α-HL subunit sequence as in SEQ ID NO: 57). Further details of the preparation and conjugation of a 1× α-HL subunit with a SpyTag peptide fusion of SEQ ID NO: 57 at N293 is described in WO2017125565A1, which is hereby incorporated by reference herein (see e.g., the α-HL subunit with C-terminal SpyTag peptide fusion of SEQ ID NO: 2 disclosed in WO2017125565A1).

Alternatively, an α-HL monomer can be engineered with cysteine residue substitutions inserted at numerous positions allowing for covalent modification of the protein through maleimide linker chemistry (see e.g., Valeva et al. (2001)). For example, the single α-HL subunit can be modified with a K46C mutation which then is easily modified with a linker allowing the use of tetrazine-trans-cyclooctene click chemistry to covalently attach a Bst2.0 variant of DNA polymerase to the heptameric 6:1 nanopore. Such an embodiment is described in U.S. Provisional Application No. 62/130,326, filed Mar. 9, 2015, and U.S. Published Patent Application No. 2017/0175183 A1, each of which is hereby incorporated by reference herein.

Other methods for attaching strand-extending enzymes to nanopores include native chemical ligation (Thapa et al., Molecules 19:14461-14483 [2014]), sortase system (Wu and Guo, J Carbohydr Chem 31:48-66 [2012]; Heck et al., Appl Microbiol Biotechnol 97:461-475 [2013]), transglutaminase systems (Dennier et al., Bioconjug Chem 25:569-578 [2014]), formylglycine linkage (Rashidian et al., Bioconjug Chem 24:1277-1294 [2013]), or other chemical ligation techniques known in the art.

4. Strand-Extending Enzymes

The nanopore threading blocker primer compositions and methods provided herein can be used with a wide range of strand-extending enzymes such as the DNA polymerases and ligases known in the art.

DNA polymerases are a family of enzymes that use single-stranded DNA as a template to synthesize the complementary DNA strand. DNA polymerases add free nucleotides to the 3′ end of a newly-forming strand resulting in extension of the new strand in the 5′-to-3′ direction. Most DNA polymerases also possess exonucleolytic activity. For example, many DNA polymerases have 3′→5′ exonuclease activity. Such multifunctional DNA polymerases can recognize an incorrectly incorporated nucleotide and use the 3′→5′ exonuclease activity to excise the incorrect nucleotide, an activity known as proofreading. Following nucleotide excision, the polymerase can re-insert the correct nucleotide and strand extension can continue. Some DNA polymerases also have 5′→3′ exonuclease activity.

DNA polymerases are used in many DNA sequencing technologies, including nanopore-based sequencing-by-synthesis. However, a DNA strand can move rapidly through the nanopore (e.g., at a rate of 1 to 5 μs per base), which can make nanopore detecting of each polymerase-catalyzed incorporation event difficult to measure and prone to high background noise, which can result in difficulties in obtaining single-nucleotide resolution. The ability to control the rate of DNA polymerase activity, as well as, increase the signal level from correct incorporation is important during sequencing-by-synthesis, particular when using nanopore detection. As shown in the Examples, the threading-blocker primer compounds of the present disclosure provide for longer read-lengths and lower percentage of deleterious threading, and thereby allow for more accurate nanopore-based nucleic acid detection and sequencing.

In some embodiments, the polymerase useful with the threading-blocker primer compounds, compositions, and methods of the present disclosure is a Pol6 DNA polymerase, or a variant of a Pol6, such as an exonuclease deficient Pol6 variant having the mutation D44A, or a Pol6 variant with an increased extension rate having the mutation Y242A and/or E585K. A range of Pol6 DNA polymerase variants having mutations providing polymerase properties useful with the various embodiments of the present disclosure are described in US patent publication nos. 2016/0222363A1, 2016/0333327 A1, 2017/0267983A1, 2018/0094249A1, 2018/0245147A1, each of which is hereby incorporated by reference herein.

Additional exemplary polymerases that may be used with the threading-blocker primer compounds, compositions, and methods of the present disclosure include the nucleic acid polymerases such as DNA polymerase (e.g., enzyme of class EC 2.7.7.7), RNA polymerase (e.g., enzyme of class EC 2.7.7.6 or EC 2.7.7.48), reverse transcriptase (e.g., enzyme of class EC 2.7.7.49), and DNA ligase (e.g., enzyme of class EC 6.5.1.1). In some embodiments, the polymerase useful with the threading-blocker primer compounds is 9° N polymerase, E. coli DNA Polymerase I, Bacteriophage T4 DNA polymerase, Sequenase, Taq DNA polymerase, 9° N polymerase (exo-)A485L/Y409V or Phi29 DNA polymerase (ϕ29 DNA Polymerase). In some embodiments, the strand extending enzyme that extends the threading-blocker primer comprises a DNA polymerase from Bacillus stearothermophilus. In some embodiments, the large fragment of DNA polymerase from B. stearothermophilus. In one embodiment, the polymerase is DNA polymerase Bst 2.0 (commercially available from New England BioLabs, Inc., Massachusetts, USA).

5. Sets of Tagged Nucleotides

Generally, the nanopore-based methods for determining the sequence of a nucleic acid using a nanopore-linked polymerase and threading-blocker primer of the present disclosure also require the use of a set of four tagged nucleotides, each of which is capable of being a substrate for the polymerase and also comprises a different nanopore-detectable tag. The tagged nucleotides useful in these methods typically comprise a compound of structural formula (IV)

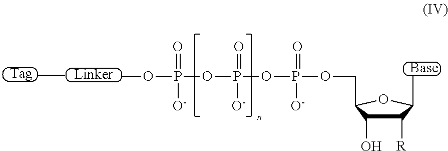

wherein, “Base” is a nucleobase selected from adenine, cytosine, guanine, thymine, and uracil; R is selected from H and OH; n is from 1 to 4; “Linker” is a linker group comprising a covalently bonded chain of 2 to 100 atoms; and “Tag” is a polymeric moiety. For example, the tagged nucleotide compound of formula (IV) can comprise a Tag selected from Table 7.

TABLE 7
Tag

-(SpC2)₁₄-(N3CEdT)₁₀-(SpC2)₆-C3
-(SpC2)₁₄-(N3CEdT)₁₀-(SpC2)₁₁-C3
-(SpC2)₁₅-(N3CEdT)₇-(SpC2)₈-C3
-(SpC2)₁₇-(N3CEdT)₁₀-(SpC2)₃-C3
-(SpC2)₁₉-(N3CEdT)₇-(SpC2)₄-C3
-(SpC2)₂₂-(N3CEdT)₇₋(SpC2)₁-C3
-(SpC2)₂₇-(N3CEdT)₇-(SpC2)₁-C3
-(SpC2)₁₇-(N3MedT)₁₀-(SpC2)₃-C3
-(SpC2)₁₇-(dT)₁₀-(SpC2)₃-03
-(SpC2)₂₃-(Tmp)₆-(SpC2)₁-C3
-(SpC2)₂₀-(Tmp)₆-(SpC2)₄-C3
-(SpC2)₁₄-(N3CEdT-Tmp)₆-(SpC2)₄-C3
-(SpC2)₁₇-(Etheno-dA)₇-(SpC2)₆-C3
-(SpC2)₂₂-(Etheno-dA)₇-(SpC2)₁-C3
-(SpC2)₁₇-(Imp)₇-(SpC2)₆-C3
-(SpC2)₁₇-(dCb)₇-(SpC2)₆-C3
-(SpC2)₂₂-(dCb)₇-(SpC2)₁-C3
-(SpC2)₂₂-(dCb)₇-(SpC2)₄-C3
-(SpC2)₁₇-(dA)₇-(SpC2)₆-C3
-(SpC2)₂₂-(dA)₇-(SpC2)₁-C3
-(SpC2)₂₁-(5MedC-PhEt)₅-(SpC2)₄-C3
-(SpC2)₁₇-(SpC2-dT)₅-(SpC2)₃-C3
-(SpC2)₁₇-(Tmp-dT)₅-(SpC2)₃-C3
-(SpC2)₁₅-(N3CEdT-5MedC-PhEt)₅-(SpC2)₅-03
-(SpC2)₁₉-(N3CEdT)₈-(SpC2)₃-C3
-TT-(SpC2)₂₈-C3
-TT-(SpC2)₁₂-(dSp)₁₀-(SpC2)₆-C3
-T₂-(SpC3)₂₈-C3
-T₂-(dSp)₂₆-T₂-C3

In the standard embodiments of methods for nanopore-based sequencing of DNA strands, the method requires a set of at least the four standard deoxy-nucleotides dA, dC, dG, and dT, wherein each different nucleotide is attached to a different tag capable of being detected upon the nucleotide being incorporated by a proximal strand extending enzyme, and furthermore wherein the each tag's nanopore detectable signal (e.g., tag current) is distinguishable from the nanopore detectable signals of each of the other three tags, thereby allowing identification of the specific nucleotide incorporated by the enzyme. Generally, each of the different tagged nucleotides in a set is distinguished by the distinctive detectable tag current signal the tag produces when it is incorporated into a new complementary strand by a strand-extending enzyme. Accordingly, a set of four tagged deoxy-nucleotides dA, dC, dG, and dT is desired that provide well-separated and resolved tag current signals when detected using a wide-pore nanopore device.

In some embodiments of the methods of the present disclosure, the method requires the use of a composition comprising a set of four tagged nucleotides (e.g., dA, dC, dG, and dT) each with a different tag, wherein each different tag results in a different detectable tag current level upon entering a nanopore of a nanopore device. For example, in some embodiments, the set of ion flow altering tagged nucleotides can comprise an oligonucleotide tag disclosed in US Pat. Publ. Nos. 2013/0244340 A1, 2013/0264207 A1, 2014/0134616 A1, 2015/0119259 A1, 2015/0368710 A1, and 2018/0057870 A1, and published International Application WO 2019/166457 A1, each of which is hereby incorporated by reference herein. Seven exemplary sets of tagged nucleotides useful in the nanopore-based methods of the present disclosure for determining a nucleic acid sequence are provided in Table 8 below.

TABLE 8

| | Avg. |
|---|---|
| | Tag Current |
| Tagged Nucleotide Set | Level |
| Set 1 | |
| dC6P-(Linker)-T₂-(SpC2)₂₈-C3 | 0.85 |
| dG6P-(Linker)-T₂-(dSp)₂₆-T₂-C3 | 0.60 |
| dT6P-(Linker)-(SpC2)₁₉-(N3CEdT)₇-(SpC2)₄-C3 | 0.35 |
| dA6P-(Linker)-(SpC2)₁₄-(N3CEdT)₁₀-(SpC2)₆-C3 | 0.15 |
| Set 2 | |
| dT6P-(Linker)-T₂-(SpC2)₂₈-C3 | 0.85 |
| dG6P-(Linker)-TT-(SpC2)12-dSp10-(SpC2)6-C3 | 0.65 |
| dC6P-(Linker)-(SpC2)₁₇-(N3MedT)₁₀-(SpC2)₃-C3 | 0.40 |
| dA6P-(Linker)-(SpC2)₁₄-(N3CEdT-Tmp)₆-(SpC2)₄-C3 | 0.05 |
| Set 3 | |
| dC6P-(Linker)-T₂-(SpC2)₂₈-C3 | 1.15 |
| dT6P-(Linker)-(SpC2)₁₉-(N3CEdT)₇-(SpC2)₄-C3 | 0.9 |
| dA6P-(Linker)-(SpC2)₁₇-(N3CEdT)₁₀-(SpC2)₃-C3 | 0.7 |
| dG6P-(Linker)-(SpC2)₁₅-(N3CEdT-5MedC-PhEt)₅-(SpC2)₅-C3 | 0.5 |
| Set 4 | |
| dA6P-(Linker)-(SpC2)₁₄-(N3CEdT)₁₀-(SpC2)₆-C3 | 0.15 |
| dC6P-(Linker)-T₂-(SpC2)₂₈-C3 | 1.15 |
| dG6P-(Linker)-T₂-(dSp)₂₆-T₂-C3 | 0.80 |
| dT6P-(Linker)-(SpC2)₁₄-(N3MedT)₁₀-(SpC2)₆-C3 | 0.35 |
| Set 5 | |
| dA6P-(Linker)-(SpC2)₁₄-(N3CEdT)₁₀-(SpC2)₆-C3 | 0.15 |
| dC6P-(Linker)-T₂-(SpC2)₂₈-C3 | 1.15 |
| dG6P-(Linker)-T₂-(dSp)₂₆-T₂-C3 | 0.80 |
| dT6P-(Linker)-(SpC2)₁₉-(N3CEdT)₇-(SpC2)₄-C3 | 0.60 |
| Set 6 | |
| dA6P-(Linker)-(SpC2)₁₄-(N3CEdT)₁₀-(SpC2)₆-C3 | 0.15 |
| dC6P-(Linker)-T₂-(SpC2)₂₈-C3 | 1.15 |
| dG6P-(Linker)-T₂-(dSp)₂₆-T₂-C3 | 0.80 |
| dT6P-(Linker)-(SpC2)₁₉-(N3CEdT)₈-(SpC2)₃-C3 | 0.67 |
| Set 7 | |
| dA6P-(Linker)-(SpC2)₁₄-(N3CEdT)₁₀-(SpC2)₁₁-C3 | 0.15 |
| dC6P-(Linker)-T₂-(SpC2)₂₈-C3 | 1.15 |
| dG6P-(Linker)-T₂-(dSp)₂₆-T₂-C3 | 0.80 |
| dT6P-(Linker)-(SpC2)₁₉-(N3CEdT)₈-(SpC2)₃-C3 | 0.67 |

As shown above in Table 8, the average tag current levels determined with wide-pore mutants for each of the four tagged nucleotides in each set are suitably well-separated to allow for good resolution and detection in a nanopore device with wide-pore nanopores. Accordingly, in some embodiments, the present disclosure provides a method wherein the set of tagged nucleotides is selected from Set 1. Set 2, Set 3, Set, 4, Set 5, Set 6, and Set 7, of Table 8. Additionally, methods and techniques for determining the nanopore detectable signal characteristics, such as tag current level and/or dwell time, are known in the art. (See e.g., US Pat. Publ. Nos. 2013/0244340 A1, 2013/0264207 A1, 2014/0134616 A1, 2015/0119259 A1, 2015/0368710 A1, and 2018/0057870 A1, and published International Application WO 2019/166457 A1, each of which is hereby incorporated by reference herein.)

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Assay of Nanopore Threading-Blocker Primers

This example illustrates assays of nanopore threading-blocker primer compounds of formulas (I) and (II) using a Pol6 polymerase-linked wide-pore mutant nanopore device. The assays demonstrate the threading-blocker primers effect of reduced deleterious template threading and increased median read lengths during nanopore sequencing measurements.

Materials and Methods

The threading-blocker primers used in the assays are shown in Table 9 below. The primers are oligonucleotides that are prepared using standard automated oligonucleotide synthesis and commercially available phosphoramidite reagents. For example, the threading-blocker primers comprising spermine as a blocking moiety are prepared using a spermine phosphoramidite reagent that incorporate the spermine into the oligonucleotide chain via a phosphodiester linkage.

The threading-blocker primers with blocking moieties attached to the via a base-modified nucleobase were prepared according to the general reaction scheme of FIG. 2. The oligonucleotide is prepared via standard automated oligonucleotide synthesis using the C8-alkyne-modified dT phosphoramidite reagent at the desired point in the sequence. The resulting oligonucleotide comprises the alkyne-modified dT nucleoside, which is then further modified using standard CuAAC azido-alkyne click-chemistry.

Briefly, as shown in FIG. 2, the oligonucleotide, 5′-DMT-(Biotin)-(Sp18)-TTTTUUUTTT-(T*)-AACGGAGGAGGAGGA-3′ was synthesized using automated oligonucleotide synthesis, then deprotected and cleaved from the synthesis resin by ammonia treatment. (As noted elsewhere herein, “T*” indicate a dT nucleoside modified with a C8-alkyne linkage at position that is introduced into the oligonucleotide using the phosphoramidite reagent, C8-alkyne-dT-CE phosphoramidite.) After the ammonia was removed under vacuum, 0.6 μmol crude DMT-protected oligo was suspended in 120 μL water. 60 μL 5M NaCl was added, and the suspension was vortexed. Separately, 150 μL 0.1M CuBr in DMSO/tBuOH (3:1) was added to 220 μL of 0.1 M THPTA in water. 300 μL of the CuBr/THPTA solution was added to the suspension of the oligonucleotide, followed with 90 μL of 10 mM aqueous solution (0.9 μmol) of K8 peptide, azidobutyryl-4NPA-εLys-(Lys)₈-NH₂. The reaction was adjusted to pH ˜7.5 with 15 μL of 1M aqueous NaHCO₃ and shaken at 25° C. for 2 days. The reaction solution was then diluted with 0.4 mL ammonia and 1 mL of 100 mg/mL NaCl. Then the suspension was purified using a 150-mg glen-pak column. The DMT group on the 5′-biotin was removed by 10-min treatment with 4% TFA, and the resulting oligonucleotide conjugate was eluted out using 0.5% ammonia in 50% aqueous acetonitrile (“ACN”). Mass spectrometric analysis showed about >95% of the desired oligonucleotide conjugate (mw ˜10358). It was concentrated under vacuum and lyophilized. To further purify, the lyophilized concentrate was then dissolved in 700 μL of 1 M triethylammonium acetate and injected over the Semi-prep C18 column (250 mm×10 mm) and eluted 3 mL/min with 5% to 25% solvent B over 40 min (solvent A=100 mM triethylammonium acetate pH 7.8, solvent B=acetonitrile). The purest fractions were combined, concentrated under vacuum, and lyophilized. It was then dissolved in 1 mL water and re-lyophilized. 50 nmol pure conjugate was obtained.

The Pol6-nanopore conjugates are embedded in membranes formed over an array of individually addressable integrated circuit chips. This nanopore device is exposed to a DNA template, a threading-blocker primer of the present disclosure, and a set of tagged nucleoside substrates selected from those listed in Table 8. In both experiments, the polymerase complex forms with the nanopore-linked polymerase, the primer, the template, and a tagged nucleotide that is complementary to the DNA template is captured and bound to the Pol6 polymerase active site, the tag polymer moiety becomes positioned in the α-HL wide-pore mutant nanopore conjugated nearby. Under the applied AC potential, the presence of the tag in the pore alters the ion flow through the nanopore relative than the O.C. current (i.e., current with no tag in the nanopore) resulting in a distinctive tag level current measured at the nanopore device electrodes. The distinctive tag current level measured as the different tag moieties enter the nanopore during Pol6 synthesis of a complementary DNA extension strand can be used to detect and identify the DNA template. Early truncation in sequencing due to template threading is determined as the number of cells that display a deep current blockage for an extended period of time that software determines to not be related to a tag binding event and at another level distinctive from the current level of the sequencing tags.

Nanopore detection system: The nanopore ion-flow measurements are performed using a nanopore array microchip comprising a CMOS microchip that has an array of approximately 8,000,000 titanium nitride electrodes within shallow wells (chip fabricated by Roche Sequencing Solutions, Santa Clara, Calif., USA). Methods for fabricating and using such nanopore array microchips can also be found in U.S. Patent Application Publication Nos. 2013/0244340 A1, US 2013/0264207 A1, US2014/0134616 A1, 2015/0368710 A1, and 2018/0057870 A1, and published International Application WO 2019/166457 A1, each of which is hereby incorporated by reference herein. Each well in the array is manufactured using a standard CMOS process with surface modifications that allow for constant contact with biological reagents and conductive salts. Each well can support a phospholipid bilayer membrane with a nanopore-polymerase conjugate embedded therein. The electrode at each well is individually addressable by computer interface. All reagents used are introduced into a simple flow cell above the array microchip using a computer-controlled syringe pump. The chip supports analog to digital conversion and reports electrical measurements from all electrodes independently at a rate of over 1000 points per second. Nanopore tag current measurements can be made asynchronously at each of 8 M addressable nanopore-containing membranes in the array at least once every millisecond (msec) and recorded on the interfaced computer.

Formation of lipid bilayer on chip: Each in a chip is first filled with a running buffer composed of 510 mM potassium acetate, 18 mM magnesium acetate, 15 mM lithium acetate, 50 mM HEPES (pH 7.8), 0.5 mM EDTA, 0.09% proclin 300 and 1% trehalose and a current applied to measure presence of buffer. The phospholipid bilayer membrane on the chip is prepared using 1,2-diphytanoyl-sn-glycero-3-phosphocholine (DPhPC, Avanti Polar Lipids). The lipid powder is dissolved in a 4:1 mixture of Silicone Oil AR20:hexadecane at a concentration of 10 mg/mL and then flown in a bolus across the wells on the chip. A thinning process then is initiated by pumping running buffer through the cis side of the array wells, thus reducing multi-lamellar lipid membranes to a single bilayer.

Insertion of α-HL-Pol6 conjugate in membrane: After the lipid bilayer forms on the wells of the array chip, 1 nM of a 6:1 wide-pore mutant α-HL-Pol6 conjugate, with prebound DNA template, all in a dilution buffer solution of 400 mM potassium acetate, 18 mM magnesium acetate, 15 mM lithium acetate, 5 mM TCEP, 50 mM HEPES, 0.5 mM EDTA, 8% trehalose, 0.001% tween 20, 0.09% proclin 300 pH 7.8, at 20° C. is added to the cis side of the chip. The nanopore-polymerase conjugate in the mixture either is electroporated or spontaneously inserts into the lipid bilayer. The non-polymerase modified α-HL subunits (i.e., the 6 subunits of the 6:1 heptamer) include the H144A mutation.

As disclosed in the results below, the wide-pore mutants disclosed in Table 6 above are used in forming the 6:1 heptamers.

The DNA template is a pUC250 circular sequence comprising the 594 bp Index 1 and Index 2 nucleotide sequences shown below.

pUC250 Index 1
(SEQ ID NO: 64)
CAGTCAGTAGAGAGAGATTGGCACGACAGGTTTCCCGACTGGAAAGCGG
GCAGTGAGCGCAACGCAATTAATGTGAGTTAGCTCACTCATTAGGCACC
CCAGGCTTTACACTTTATGCTTCCGGCTCGTATGTTGTGTGGAATTGTG
AGCGGATAACAATTTCACACAGGAAACAGCTATGACCATGATTACGCCA
AGCTTGCATGCCTGCAGGTCGACTCTAGAGGATCCCCGGGTACCGAGCT
CGAATTCACTGGCCGTCGTTTTACAATCTCTCTCAAAAACGGAGGAGGA
GGACAGTCAGTAGAGAGAGATTGTAAAACGACGGCCAGTGAATTCGAGC
TCGGTACCCGGGGATCCTCTAGAGTCGACCTGCAGGCATGCAAGCTTGG
CGTAATCATGGTCATAGCTGTTTCCTGTGTGAAATTGTTATCCGCTCAC
AATTCCACACAACATACGAGCCGGAAGCATAAAGTGTAAAGCCTGGGGT
GCCTAATGAGTGAGCTAACTCACATTAATTGCGTTGCGCTCACTGCCCG
CTTTCCAGTCGGGAAACCTGTCGTGCCAATCTCTCTCAAAAACGGAGGA
GGAGGA
pUC250 Index 2
(SEQ ID NO: 65)
CAGTCAGTAGAGAGAGATTGTAAAACGACGGCCAGTGAATTCGAGCTCG
GTACCCGGGGATCCTCTAGAGTCGACCTGCAGGCATGCAAGCTTGGCGT
AATCATGGTCATAGCTGTTTCCTGTGTGAAATTGTTATCCGCTCACAAT
TCCACACAACATACGAGCCGGAAGCATAAAGTGTAAAGCCTGGGGTGCC
TAATGAGTGAGCTAACTCACATTAATTGCGTTGCGCTCACTGCCCGCTT
TCCAGTCGGGAAACCTGTCGTGCCAATCTCTCTCAAAAACGGAGGAGGA
GGACAGTCAGTAGAGAGAGATTGGCACGACAGGTTTCCCGACTGGAAAG
CGGGCAGTGAGCGCAACGCAATTAATGTGAGTTAGCTCACTCATTAGGC
ACCCCAGGCTTTACACTTTATGCTTCCGGCTCGTATGTTGTGTGGAATT
GTGAGCGGATAACAATTTCACACAGGAAACAGCTATGACCATGATTACG
CCAAGCTTGCATGCCTGCAGGTCGACTCTAGAGGATCCCCGGGTACCGA
GCTCGAATTCACTGGCCGTCGTTTTACAATCTCTCTCAAAAACGGAGGA
GGAGGA

Nanopore ion flow measurements: After insertion of the complex into the membrane, the solution on the cis side is replaced by an osmolarity buffer: 400 mM potassium acetate, 18 mM magnesium acetate, 15 mM lithium acetate, 5 mM TCEP, 50 mM HEPES, 0.5 mM EDTA, 0.09% proclin 300 pH 7.8. Sequencing solution containing a set of the 4 different nucleotide substrates is added (3 μM of each sequencing tag). 500 μM of each of the set of the 4 different nucleotide substrates is added. The trans side buffer solution is: 400 mM potassium acetate, 18 mM magnesium acetate, 15 mM lithium acetate, 5 mM TCEP, 50 mM HEPES, 0.5 mM EDTA, 8% trehalose, 0.001% tween 20, 0.09% proclin 300 pH 7.8. These buffer solutions are used as the electrolyte solutions for the nanopore ion flow measurements. A Pt/Ag/AgCl electrode setup is used and an AC current of a 180, 210, 220, or 280 mV pk-to-pk waveform applied at 976 Hz or 1429 Hz. AC current has certain advantages for nanopore detection as it allows for the tag to be repeatedly directed into and then expelled from the nanopore thereby providing more opportunities to measure signals resulting from the ion flow through the nanopore. Also, the ion flow during the positive and negative AC current cycles counteract each other to reduce the net rate of ion depletion from the cis side, and possible detrimental effects on signals resulting from this depletion.

Briefly, the nanopore assay of the threading-blocker primers is carried out using an array of wide-pore mutant α-HL nanopores each conjugated to a Pol6 polymerase variant, such as an exonuclease deficient Pol6 variant with increased extension rate, as described in US patent publication nos. 2016/0222363A1, 2016/0333327A1, 2017/0267983A1, 2018/0094249A1, and 2018/0245147A1, each of which is hereby incorporated by reference herein.

The tag current level signal representing the distinct altered ion-flow event resulting from each different polymer moiety tag is observed as the tagged nucleotide is captured by the α-HL-Pol6 nanopore-polymerase conjugates primed with the DNA template. Plots of these events are recorded over time and analyzed. Generally, events that last longer than 10 ms indicate productive tag capture coincident with polymerase incorporation of the correct base complementary to the template strand.

Read length and percent threaded properties of the threading-blocker primers were assessed in nanopore assay under nanopore sequencing conditions as described herein. At the completion of sequencing and analysis, the median read length based on the high quality reads was gathered and the percent of total high quality reads that ended prematurely was determined as a fraction of early terminations in sequencing of high quality reads over all high quality reads.

Results of nanopore assays showing the read length and % threaded are shown for a control primer and a variety of threading-blocker primers in Table 9 below. The reading-blocker primers tend to exhibit substantially increased read lengths and decreased % threaded values relative to the control primer.

TABLE 9

| | read | |
|---|---|---|
| | length | % |
| Threading Blocker Primer | (bp) | threaded |
| 5′-(Biotin)-(Sp18)-TTTTUUUTTTAACGGAGGAGGAGGA-3′ (SEQ ID NO: 66) [Control] | <1000 | 50-60% |
| 5′-(Biotin)-(Sp18)-TTTTUUUTT-(spermine)-TAACGGAGGAGGAGGA-3′ | <1000 | 50-60% |
| (SEQ ID NO: 67) | | |
| 5′-(Biotin)-(Sp18)-TTTTUUUT-(spermine)₂-TAACGGAGGAGGAGGA-3′ | >2500 | <10% |
| (SEQ ID NO: 68) | | |
| 5′-(Biotin)-(Sp18)-TTTTUUUTT-(dU-[perylene])-TAACGGAGGAGGAGGA-3′ | <1000 | 40-50% |
| (SEQ ID NO: 69) | | |
| 5′-(Biotin)₂-(Sp18)-TTTUUUTT-(dU-[ethynyl])-TAACGGAGGAGGAGGA-3′ | <1000 | 50-60% |
| (SEQ ID NO: 70) | | |
| 5′-(Biotin)₂-(Sp18)-TTTUUUTT-(dU-[perylene])-TAACGGAGGAGGAGGA-3′ | <1000 | 40-50% |
| (SEQ ID NO: 71) | | |
| 5′-(Biotin)-(Sp18)-TTTTUUUTTT-(spermine)₂-TAACGGAGGAGGAGGA-3′ | >1500 | <10% |
| (SEQ ID NO: 72) | | |
| 5′-(Biotin)-(Sp18)-TTTTUUU-(Sp1 8)-(spermine)₂-TAACGGAGGAGGAGGA-3′ | >1500 | <10% |
| (SEQ ID NO: 73) | | |
| 5′-(Biotin)-(Sp18)-TTTTUUUTTT-(ethylenediamine)₅-TAACGGAGGAGGAGGA-3′ | <1000 | >70% |
| (SEQ ID NO: 74) | | |
| 5′-(Biotin)-(Sp18)-TTTTUUUTTT-(ethylenediamine)₆-TAACGGAGGAGGAGGA-3′ | <1000 | >70% |
| (SEQ ID NO: 75) | | |
| 5′-(Biotin)-(Sp18)-TTTTUUUTTTA-(dU-[pyrene])₂-AACGGAGGAGGAGGA-3′ | <1000 | 40-50% |
| (SEQ ID NO: 76) | | |
| 5′-(Biotin)-(Sp18)-TTTTUUUTT-(spermine)₅-TAACGGAGGAGGAGGA-3′ | >2500 | 10-15% |
| (SEQ ID NO: 77) | | |
| 5′-(Biotin)-(Sp18)-TUUTTTTUU-(spermine)₃-TAACGGAGGAGGAGGA-3′ | <1000 | <10% |
| (SEQ ID NO: 78) | | |
| 5′-(Biotin)-(Sp18)-TUUTTTTTUU-(spermine)₂-TAACGGAGGAGGAGGA-3′ | 2500 | <10% |
| (SEQ ID NO: 79) | | |
| 5′-(Biotin)-(Sp18)-TTTTUUUUUU-(spermine)₂-TAACGGAGGAGGAGGA-3′ | >2500 | <10% |
| (SEQ ID NO: 80) | | |
| 5′-(Biotin)-(Sp18)-TTTTUUUTT-(spermine)₃-TAACGGAGGAGGAGGA-3′ | >2500 | <10% |
| (SEQ ID NO: 81) | | |
| 5′-(Biotin)-(Sp18)-TTTTUUTTTTTUUT-(cholesteryl-TEG)-TAACGGAGGAGGAGGA-3′ | <1000 | 15-25% |
| (SEQ ID NO: 82) | | |
| 5′-(Biotin)-(Sp18)-TTTTUUUTTT-(T*-[Phe(4-NO₂)-ϵLys-(Lys)₁₂])-AACGGAGGAGGAGGA-3′ | >2500 | 10-15% |
| (SEQ ID NO: 83) | | |
| 5′-(Biotin)-(Sp18)-TTTTUUUTTT-(T*[PAMAM Gent1 amino])-AACGGAGGAGGAGGA-3′ | >2500 | 10-15% |
| (SEQ ID NO: 84) | | |
| 5′-(DesthiobiotinTEG)-(Sp18)2-(T*-[Phe(4-NO₂)-ϵLys-(Lys)₈])-AACGGAGGAGGAGGA-3′ | >3000 | <15% |
| (SEQ ID NO: 85) | | |
| 5′-(DesthiobiotlnTEG)-(Sp18)3-(T*-[Phe(4-NO₂)-ϵLys-(Lys)₈])-AACGGAGGAGGAGGA-3′ | >3000 | <15% |
| (SEQ ID NO: 86) | | |
| 5′-(DesthiobiOtinTEG)-(Sp18)3-(T*-[Phe(4-NO₂)-ϵLys-(Lys)₁₂])-AACGGAGGAGGAGGA-3′ | >3000 | <15% |
| (SEQ ID NO: 87) | | |

1. A composition comprising a compound of formula (I):
5′-[Blocking Moiety]-[Primer]-3′ (I)
wherein,
the Blocking Moiety comprises a poly-cationic group, a bulky group, or a base-modified nucleoside, wherein the base-modified nucleoside comprises a poly-cationic group or a bulky group attached to the nucleoside base; and
the Primer comprises an oligonucleotides capable of priming polymerization of a copy strand by a polymerase linked to a nanopore.

2. The composition of claim 1, wherein the compound of formula (I) comprises a compound of formula selected from a group consisting of (Ia): 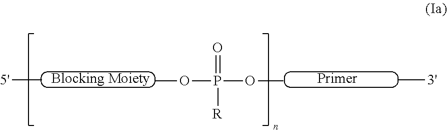
wherein,
n is 1 to 10; and
R is independent selected from O⁻, S⁻, CH₃, and H;
(Ib): 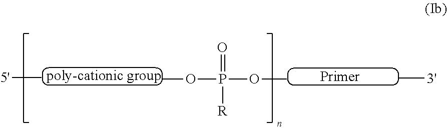
wherein,
n is 1 to 10; and
R is independently selected from O⁻, CH₃, and H;
(Ic): 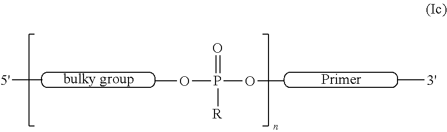
wherein,
n is 1 to 10; and
R is independently selected from O⁻, S⁻, CH₃, and H;
(Id): 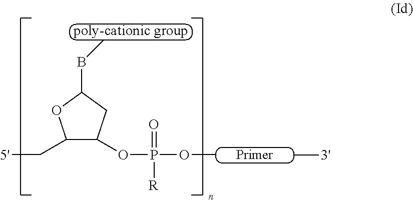
wherein,
n is 1 to 10;
B is a modified nucleobase; and
R is independently selected from O⁻, S⁻, CH₃, and H;
and (Ie): 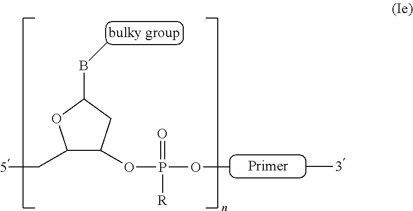
wherein,
n is 1 to 10;
B is a modified nucleobase; and
R is independently selected from O⁻, S⁻, CH₃, and H.

3. The composition of claim 1, wherein the compound of formula (I) further comprises a biotin tag attached to the 5′-end of the Blocking Moiety.

4. A composition comprising a compound of formula (II):
5′-[Biotin Tag]-[Blocking Moiety]-[Primer]-3′ (II)
wherein,
the Biotin Tag comprises a biotin tag;
the Blocking Moiety comprises a poly-cationic group, a bulky group, or a base-modified nucleoside, wherein the base-modified nucleoside comprises a poly-cationic group or a bulky group attached to the nucleoside base; and
the Primer comprises an oligonucleotide capable of priming polymerization of a copy strand by a polymerase linked to a nanopore.

5. The composition of claim 4, wherein the compound of formula (II) comprises a compound of formula selected from
(IIa): 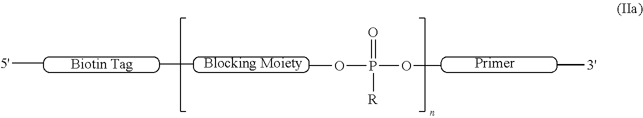
wherein,
n is 1 to 10; and
R is independently selected from O⁻, S⁻, CH₃, and H;
(IIb): 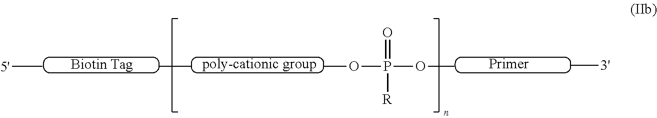
wherein,
n is 1 to 10; and
R is independently selected from O⁻, S⁻, CH₃, and H;
(IIc): 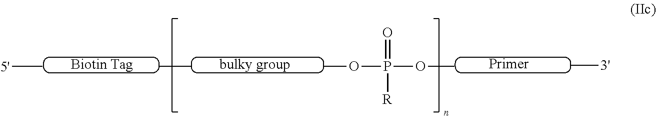
wherein,
n is 1 to 10; and
R is independently selected from O⁻, S⁻, CH₃, and H;
(IId): 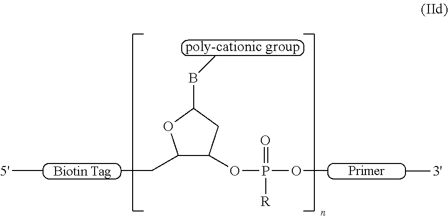
wherein,
n is 1 to 10;
B is a modified nucleobase; and
R is independently selected from O⁻, S⁻, CH₃, and H; and
(IIe): 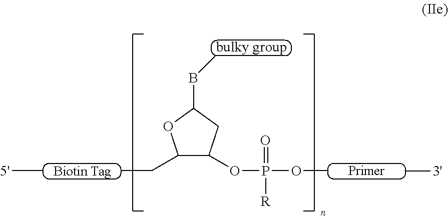
wherein,
n is 1 to 10;
B is a modified nucleobase; and
R is independently selected from O⁻, S⁻, CH₃, and H.

6. The composition of claim 3, wherein the biotin tag comprises a structure of formula (III):
B-L-[(N)ₓ—(U)_{y}—(N)_{z}]_{w} (III)
wherein,
the B is biotin or desthiobiotin;
the L is a linker;
the N is a nucleotide;
the U is deoxyuridine; and
the x and the z are at least 1; they is at least 3; and the w is 0 or 1.

7. The composition of claim 3, wherein the biotin tag comprises a biotin moiety and a linker moiety or a desthiobiotin moiety and a linker moiety, wherein the linker moiety attaches to the 5′-end of the Blocking Moiety.

8. The composition of any one of claims 1-7, wherein the Blocking Moiety comprises a poly-cationic group.

9. The composition of claim 8, wherein the poly-cationic group is selected from spermine, spermidine, Phe(4-NO₂)-εLys-(Lys)₈], [Phe(4-NO₂)-εLys-(Lys)₁₂], [(Lys)₈-εLys-Phe(4-NO₂)], [(Lys)₁₂-εLys-Phe(4-NO₂)], [PAMAM Gen1 amino], poly(ethylenediamine), poly(propylenediamine), poly(allylamine), an oligomer of a cationic amino acid, and an oligomer of a cationic aminoalkyl.

10. The composition of claim 8, wherein the poly-cationic group is an oligomer of a cationic amino acid selected from an oligomer of lysine, ε-lysine, ornithine, (aminoethyl)glycine, arginine, histidine, methyllysine, dimethyllysine, trimethyllysine, and/or aminoproline.

11. The composition of claim 8, wherein the poly-cationic group is an oligomer of spermine groups.

12. The composition of any one of claims 1-7, wherein the Blocking Moiety comprises a bulky group.

13. A nanopore composition comprising:
a membrane having an electrode on a cis side of the membrane and having an electrode on a trans side of the membrane;
a nanopore with its pore extending through the membrane;
an active polymerase situated adjacent to the nanopore;
an electrolyte solution comprising ions in contact with both electrodes; and
a compound of formula (I), a compound of formula (II), or composition of any one of claims 1-7.

14. A kit comprising:
a nanopore device, wherein the nanopore device comprises a membrane having an electrode on a cis side and having an electrode on a trans side of the membrane, a nanopore with its pore extending through the membrane, and an active polymerase situated adjacent to the nanopore;
a set of four tagged nucleotides; and
a compound of formula (I), a compound of formula (II), or a composition of any one of claims 1-7.

15. A method for determining the sequence of a nucleic acid, wherein the method comprises the following steps:
(a) providing a nanopore composition comprising: a membrane, an electrode on the cis side, an electrode on the trans side of the membrane, a nanopore with its pore extending through the membrane, an active polymerase situated adjacent to the nanopore, an electrolyte solution comprising ions in contact with both electrodes, and a compound of formula (I), a compound of formula (II), or a composition of any one of claims 1-7;
(b) contacting the nanopore composition of (a) with: (i) a nucleic acid; and (ii) a set of four tagged nucleotides, each capable of acting as polymerase substrate, and each linked to a different tag which results in a different altering of the flow of ions through the nanopore when the tag enters the nanopore; and
(c) detecting the different altering of the flow of ions resulting from the entry of the different tags in the nanopore over time and correlating to each of the different compounds incorporated by the polymerase which are complementary to the nucleic acid sequence, and thereby determining the nucleic acid sequence.
